(19) United States
(12) Patent Application Publication
(10) Pub. No.: US 2013/0225496 A1
(43) Pub. Date: Aug. 29, 2013

(54) Albumin Variants

(71) Applicants: Andrew Plumridge, Derbyshire (GB); Jason Cameron, Nottinghamshire (GB); Inger Sandlie, Oslo (NO); Jan Terje Andersen, Oslo (NO); Esben Peter Friis, Herlev (DK)

(72) Inventors: Andrew Plumridge, Derbyshire (GB); Jason Cameron, Nottinghamshire (GB); Inger Sandlie, Oslo (NO); Jan Terje Andersen, Oslo (NO); Esben Peter Friis, Herlev (DK)

(73) Assignee: Novozymes Biopharma DK A/S, Bagsvaerd (DK)

(21) Appl. No.: 13/882,653

(22) PCT Filed: Nov. 1, 2011
(86) PCT No.: PCT/EP2011/069188

§ 371 Date: Apr. 30, 2013

(30) Foreign Application Priority Data
Nov. 1, 2010 (EP) PCT/EP2010/066572
May 5, 2011 (EP) 11164955.4

(51) Int. Cl. C07K 14/76; A61K 47/48; C07K 14/765

(52) U.S. Cl. CPC C07K 14/76; C07K 14/765; A61K 47/48284
USPC 514/15.2; 530/362; 530/363; 536/23.5; 536/23.4; 514/44 R; 435/320.1; 435/254.21; 435/69.1

(57) ABSTRACT

The invention relates to variants of albumin. The invention also relates to polynucleotides encoding the variants; nucleic acid constructs, vectors, and host cells comprising the polynucleotides; and methods of preparing the variants and to methods of using the variants.

### REFERENCE TO A SEQUENCE LISTING

This application contains a Sequence Listing in computer readable form, which is incorporated herein by reference.

### BACKGROUND OF THE INVENTION

1. Field of the Invention

The invention relates to variants of albumin or fragments thereof or fusion polypeptides comprising variant albumin or fragments thereof having a change in half-life compared with the albumin, fragment thereof or fusion polypeptide comprising albumin or a fragment thereof from which the variant is derived or compared with another reference albumin. The invention particularly relates to molecules of albumin in which the molecule is based on the amino acid sequence of a first albumin and the extreme C terminus of that first albumin is replaced with an extreme C terminal sequence of a second albumin. The invention allows tailoring of half-life of an albumin to the requirements and desires of a user or application.

2. Description of the Related Art

Albumin is a protein naturally found in the blood plasma of mammals where it is the most abundant protein. Albumin has important roles in maintaining the desired osmotic pressure of the blood and also in the transport of various substances in the bloodstream.

Albumin contains three domains: Domain I (DI), Domain II (DII) and Domain III (DIII). Albumins have been characterized from many species, including human, pig, mouse, rat, rabbit and goat, and they share a high degree of sequence and structural homology.

Albumin binds in vivo to its receptor, the neonatal Fc receptor (FcRn) “the Brambell receptor” and this interaction is known to be important for the plasma half-life of albumin. FcRn is a membrane bound protein, expressed in many cell and tissue types. FcRn has been found to salvage albumin from intracellular degradation (Roopenian D. C. and Akilesh, S. (2007), Nat. Rev. Immunol 7, 715-725.). FcRn is a bifunctional molecule that contributes to maintaining a high level of IgGs and albumin in serum in mammals such as human beings.

Whilst the FcRn-immunoglobulin (IgG) interaction has been characterized in the prior art, the FcRn-albumin interaction is less well characterized. The major FcRn binding site is localized within DIII (381-585) (Andersen et al (2010) Clinical Biochemistry 43, 367-372). Data indicates that IgG and albumin bind non-cooperatively to distinct sites on FcRn (Andersen et al. (2006), Eur. J. Immunol. 36, 3044-3051; Chaudhury et al. (2006), Biochemistry 45, 4983-4990.).

It is known that mouse FcRn binds IgG from mice and humans whereas human FcRn appears to be more discriminating (Ober et al. (2001) Int. Immunol 13, 1551-1559). Andersen et al. ((2010), Journal of Biological Chemistry 285(7):4826-36) describes the affinity of human and mouse FcRn for both mouse and human albumin (all possible combinations). No binding of albumin from either species was observed at physiological pH, to either receptor. At acidic pH, a 100-fold difference in binding affinity was observed. In all cases, binding of albumin and IgG from either species to both receptors were additive.

Human serum albumin (HSA) has been well characterized as a polypeptide of 585 amino acids, the sequence of which can be found in Peters, T., Jr. (1996) All About Albumin: Biochemistry, Genetics and Medical Applications pp 10, Academic Press, Inc., Orlando (ISBN 0-12-552110-3). It has a characteristic binding to its receptor FcRn, where it binds at pH 6.0 but not at pH 7.4.

The plasma half-life of HSA has been found to be approximately 19 days. A natural variant having lower plasma half-life has been identified (Peach, R. J. and Brennan, S. O., (1991) Biochim Biophys Acta 1097:49-54) having the substitution D494N. This substitution generated an N-glycosylation site in this variant, which is not present in the wild-type albumin. It is not known whether the glycosylation or the amino acid change is responsible for the change in plasma half-life.

Due to its long plasma half-life, albumin has been suggested for use in drug delivery. Albumin has been conjugated to pharmaceutically beneficial compounds (WO 2000/69902A), and it was found that the conjugate maintained the long plasma half-life of albumin. Therefore the resulting plasma half-life of the conjugate was generally considerably longer than the plasma half-life of the beneficial therapeutic compound alone.

Furthermore, albumin has been genetically fused to therapeutically beneficial peptides (WO 2001/79271 A and WO 2003/59934 A) with the typical result that the protein fusion has the activity of the therapeutically beneficial peptide and a considerably longer plasma half-life than the plasma half-life of the therapeutically beneficial peptide alone.

Otagiri et al (2009), Biol. Pharm, Bull. 32(4), 527-534, discloses that 77 albumin variants are known, of these 25 have mutations in domain III. A natural variant lacking the C-terminal 175 amino acids at the carboxy terminus has been shown to have a reduced half-life (Andersen et al (2010), Clinical Biochemistry 43, 367-372). Iwao et al (2007) studied the half-life of naturally occurring human albumin variants using a mouse model, and found that K541 E and K560E had reduced half-life, E501K and E570K had increased half-life and K573E had almost no effect on half-life (Iwao, et al (2007) B.B.A. Proteins and Proteomics 1774, 1582-1590).

Galliano et al (1993) Biochim. Biophys. Acta 1225, 27-32 discloses a natural variant E505K. Minchiotti et al (1990) discloses a natural variant K536E. Minchiotti et al (1987) Biochim. Biophys. Acta 916, 411-418 discloses a natural variant K574N. Takahashi et al (1987) Proc. Natl. Acad. Sci. USA 84, 4413-4417, discloses a natural variant D550G. Carlson et al (1992). Proc. Nat. Acad. Sci. USA 89, 8225-8229, discloses a natural variant D550A.

Albumin has the ability to bind a number of ligands and these become associated (associates) with albumin. This property has been utilized to extend the plasma half-life of drugs having the ability to non-covalently bind to albumin. This can also be achieved by binding a pharmaceutically beneficial compound, which has little or no albumin binding properties, to a moiety having albumin binding properties. See review article and reference therein, Kratz (2008) Journal of Controlled Release 132, 171-183.

Albumin is used in preparations of pharmaceutically beneficial compounds, in which such a preparation maybe for example, but not limited to, a nano particle or micro particle of albumin. In these examples the delivery of a pharmaceutically beneficial compound or mixture of compounds may benefit from alteration in the albumin's affinity to its receptor where the beneficial compound has been shown to associate with albumin for the means of delivery.

It is not clear what determines the plasma half-life of the formed associates (for example but not limited to Levemir®, Kurtzhals P et al. Biochem. J. 1995; 312:725-731), conjugates or fusion polypeptides but it appears to be a result of the combination of the albumin and the selected pharmaceutically beneficial compound/polypeptide. It would be desirable to be able to control the plasma half-life of given albumin conjugates, associates or albumin fusion polypeptides so that a longer or shorter plasma half-life can be achieved than given by the components of the association, conjugation or fusion, in order to be able to design a particular drug according to the required in vivo levels for the clinical indication intended to be treated.

Albumin is known to accumulate and be catabolised in tumours, it has also been shown to accumulate in inflamed joints of rheumatoid arthritis sufferers. See review article and reference therein, Kratz (2008) Journal of Controlled Release 132, 171-183. It is envisaged that HSA variants with increased affinity for FcRn would be advantageous for the delivery of pharmaceutically beneficial compounds.

It may even be desirable to have variants of albumin that have little or no binding to FcRn in order to provide shorter half-lives or controlled serum pharmacokinetics as described by Kenanova et al (2009) J. Nucl. Med.; 50 (Supplement 2):1582).

International patent application PCT/EP10/066,572 (WO 2011/051489) discloses a first class of variant albumins having modulated (i.e. increased or decreased) binding affinity to FcRn receptor due to the presence of one or more point mutations in the albumin sequence. International patent application PCT/EP2011/055577 (WO 2011/124718) discloses a second class of variant albumins having modulated binding affinity to FcRn receptor, the variants comprise domain III of an albumin with one or more other domains of albumin and optionally include one or more point mutations.

The present invention provides a further class of variants having modulated binding affinity to the FcRn receptor and, through provision of a range of molecules (albumin variants), allows binding affinity (and therefore) half-life to be tailored according to requirements. Such tailoring may range from a large increase in half-life to a small increase in half-life, a small decrease in half-life to a large decrease in half-life. The albumin moiety or moieties may therefore be used to tailor the half-life of fusion polypeptides, conjugates, associates, nanoparticles, microparticles and compositions comprising the albumin moiety.

### SUMMARY OF THE INVENTION

The invention provides a new class of variants of a parent albumin with improved properties compared with the parent (or reference). In particular the invention provides variants of a parent albumin having altered plasma half-life compared with its parent (or reference) and/or altered binding affinity to FcRn.

The invention also relates to isolated polynucleotides encoding the variants; nucleic acid constructs, vectors, and host cells comprising the polynucleotides; and methods of producing the variants. Furthermore, the invention relates to uses of the variants, fusions of the variants, conjugations of the variants, associates of the variants and to compositions, such as pharmaceutical compositions, comprising a variant, fusion, conjugation or associate according to the invention.

### BRIEF DESCRIPTION OF THE FIGURES

FIG. 1: Multiple alignment of amino acid sequences of (i) full length mature HSA (Hu_{—}1_{—}2_{—}3), (ii) an albumin variant comprising domain I and domain III of HSA (Hu_{—}1_{—}3), (iii) an albumin variant comprising domain II and domain III of HSA (Hu_{—}2_{—}3), (iv) full-length Macaca mulatta albumin (Mac_mul), (v) full-length Rattus norvegicus albumin (Rat) and (vi) full-length Mus musculus albumin (Mouse). Positions 500, 550 and 573 (relative to full length HSA) are indicated by arrows.

FIG. 2: Multiple alignment of amino acid sequence of mature albumin from human, sheep, mouse, rabbit and goat and immature albumins from chimpanzee (“Chimp”), macaque, hamster, guinea pig, rat, cow, horse, donkey, dog, chicken, and pig. The Start and End amino acids of domains 1, 2 and 3 (as defined by Dockal et al (The Journal of Biological Chemistry, 1999, Vol. 274(41): 29303-29310)) are indicated with respect to mature human albumin.

FIG. 3: Schematic diagram of a first albumin, variant or fragment thereof (1) comprising an N(N1) and C(C1) region; a second albumin, variant or fragment thereof (2) comprising an N (N2) and C(C2) and a polypeptide (3) comprising the N-terminal region (N1) of the first albumin, albumin variant or fragment thereof and the C-terminal region (C2) of a second albumin, albumin variant or fragment thereof.

FIG. 4: Representative SPR sensorgrams showing binding of C-terminal truncated HSA variants to shFcRn. 10 μM of each variant was injected over immobilized shFcRn (2000 RU) at pH 6.0.

FIG. 5: Bar chart showing the binding affinity of C-terminal truncated HSA variants relative to wild-type (WT) HSA. KD(b): kinetic rate constants obtained using a simple first-order (1:1) bimolecular interaction model (kinetic values represent the average of duplicates), KD(c): steady state affinity constants obtained using equilibrium (Req) binding model supplied by BIAevaluation 4.1 software.

### DETAILED DESCRIPTION OF THE INVENTION

The invention relates to isolated variants of albumin or fragments thereof of a parent albumin in which the variant comprises (i) an N-terminal region of a first (parent) albumin, albumin variant or fragment thereof; and (ii) a C-terminal region of a second (parent) albumin, albumin variant or fragment thereof in which: (a) the N-terminal of the first (parent) albumin, albumin variant or fragment thereof comprises the amino acids of the molecule from which it is derived except the C-terminal 1 to 205 amino acids; and (b) the C-terminal of the second (parent) albumin, albumin variant or fragment thereof comprises the C-terminal 1 to 205 amino acids of the second (parent) albumin, albumin variant or fragment thereof; and

(c) the polypeptide has an altered half-life and/or altered FcRn-binding affinity compared with the first (parent) albumin, albumin variant or fragment thereof or to another reference such as HSA.

Furthermore the invention relates to fusions, conjugations (also referred to as conjugates) and associations of the albumin variant or fragment thereof. The invention also relates to polynucleotides, including vectors such as plasmids, encoding the albumin, fragment or fusion and to host cells comprising such polynucleotides. Methods of producing and/or using the albumin variants, fragments, fusions, conjugations, associations, polynucleotides, vectors and host cells are also included in the invention.

The following definitions apply to the invention and may be further defined throughout the specification:

### DEFINITIONS

Variant: The term “variant” means a polypeptide derived from a parent albumin by one or more alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, preferably 1-3 amino acids immediately adjacent to an amino acid occupying a position.

Mutant: The term “mutant” means a polynucleotide encoding a variant.

Wild-Type Albumin: The term “wild-type” (WT) albumin means albumin having the same amino acid sequence as naturally found in an animal, such as in a human being.

Parent or Parent albumin The term “parent” or “parent albumin” means an albumin to which an alteration is made by the hand of man to produce the albumin variants of the invention.

The parent may be a naturally occurring (wild-type) polypeptide or an allele thereof, or even a variant thereof such as a variant described in PCT/EP2010/066572 (WO 2011/066572) or a variant or derivative described in PCT/EP2011/055577 (WO 2011/124718). In this specification there may be one or two parent albumins.

FcRn and shFcRn: The term “FcRn” means the human neonatal Fc receptor (FcRn). shFcRn is a soluble recombinant form of FcRn. hFcRn is a heterodimer of SEQ ID NO: 30 (truncated heavy chain of the major histocompatibility complex class I-like Fc receptor (FCGRT)) and SEQ ID NO: 31 (beta-2-microglobulin). Together, SEQ ID NO: 30 and 31 form hFcRn. shFcRn, for example GST-tagged shFcRn, may be made according to the methods described below.

Isolated variant: The term “isolated variant” means a variant that is modified by the hand of man and separated completely or partially from at least one component with which it naturally occurs. The variant may be at least 1% pure, e.g. at least 5% pure, at least 10% pure, at least 20% pure, at least 40% pure, at least 60% pure, at least 80% pure, at least 90% pure, at least 95% pure, at least 96% pure, at least 97% pure, at least 98% pure or at least 99% pure as determined by SDS-PAGE or GP-HPLC.

Substantially pure variant: The term “substantially pure variant” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the variant is at least 92% pure, e.g. at least 94% pure, at least 95% pure, at least 96% pure, at least 97% pure, at least 98% pure, at least 99%, at least 99.5% pure, and 100% pure by weight of the total polypeptide material present in the preparation. The variants of the invention are preferably in a substantially pure form. This can be accomplished, for example, by preparing the variant by well known recombinant methods and by purification methods.

Mature polypeptide: The term “mature polypeptide” means a polypeptide in its final form following translation and any post-translational modifications, such as N-terminal processing, C-terminal truncation, glycosylation, phosphorylation, etc. The mature polypeptide may comprise or consist of a polypeptide according to the first or fourth aspect of the invention with the inclusion of any post-translational modifications.

Mature polypeptide coding sequence: The term “mature polypeptide coding sequence” means a polynucleotide that encodes a mature albumin polypeptide.

Sequence Identity: The relatedness between two amino acid sequences or between two nucleotide sequences is described by the parameter “sequence identity”.

For purposes of the invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labelled “longest identity” (obtained using the -nobrief option) is used as the percent identity and is calculated as follows:

(Identical Residues×100)/(Length of Alignment−Total Number of Gaps in Alignment).

For purposes of the invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Alternative alignment tools can also be used, for example MUSCLE as described herein.

Fragment: The term “fragment” means a polypeptide having one or more (several) amino acids deleted from the amino and/or carboxyl terminus of an albumin and/or an internal region of albumin that has retained the ability to bind to FcRn. Fragments may consist of one uninterrupted sequence derived from HSA or it may comprise two or more sequences derived from HSA. The fragments according to the invention have a size of more than approximately 20 amino acid residues, preferably more than 30 amino acid residues, more preferred more than 40 amino acid residues, more preferred more than 50 amino acid residues, more preferred more than 75 amino acid residues, more preferred more than 100 amino acid residues, more preferred more than 200 amino acid residues, more preferred more than 300 amino acid residues, even more preferred more than 400 amino acid residues and most preferred more than 500 amino acid residues.

Allelic variant: The term “allelic variant” means any of two or more alternative forms of a gene occupying the same chromosomal locus. Allelic variation arises naturally through mutation, and may result in polymorphism within populations. Gene mutations (alterations) can be silent (no change in the encoded polypeptide) or may encode polypeptides having altered amino acid sequences. An allelic variant of a polypeptide is a polypeptide encoded by an allelic variant of a gene.

Isolated polynucleotide: The term “isolated polynucleotide” means a polynucleotide that is modified by the hand of man. The isolated polynucleotide may be at least 1% pure, e.g. at least 5% pure, at least 10% pure, at least 20% pure, at least 40% pure, at least 60% pure, at least 80% pure, at least 90% pure, and at least 95% pure, as determined by agarose electrophoresis. The polynucleotides may be of genomic, cDNA, RNA, semi-synthetic, synthetic origin, or any combinations thereof.

Substantially pure polynucleotide: The term “substantially pure polynucleotide” means a polynucleotide preparation free of other extraneous or unwanted nucleotides and in a form suitable for use within genetically engineered polypeptide production systems. Thus, a substantially pure polynucleotide contains at most 10%, at most 8%, at most 6%, at most 5%, at most 4%, at most 3%, at most 2%, at most 1%, and at most 0.5% by weight of other polynucleotide material with which it is natively or recombinantly associated. A substantially pure polynucleotide may, however, include naturally occurring 5′- and 3′-untranslated regions, such as promoters and terminators. It is preferred that the substantially pure polynucleotide is at least 90% pure, e.g. at least 92% pure, at least 94% pure, at least 95% pure, at least 96% pure, at least 97% pure, at least 98% pure, at least 99% pure, and at least 99.5% pure by weight. The polynucleotides of the invention are preferably in a substantially pure form.

Coding sequence: The term “coding sequence” means a polynucleotide, which directly specifies the amino acid sequence of its translated polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the invention.

Control sequences: The term “control sequences” means all components necessary for the expression of a polynucleotide encoding a variant of the invention. Each control sequence may be native or foreign to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences within the coding region of the polynucleotide encoding a variant.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to additional nucleotides that provide for its expression.

Host cell: The term “host cell” means any cell type that is susceptible to transformation, transfection, transduction, and the like with a nucleic acid construct or expression vector comprising a polynucleotide of the invention. The term “host cell” encompasses any progeny of a parent cell that is not identical to the parent cell due to mutations that occur during replication.

Plasma half-life: Plasma half-life is ideally determined in vivo in suitable individuals. However, since it is time consuming and expensive and there are inevitable ethical concerns connected with doing experiments in animals or man it is desirable to use an in vitro assay for determining whether plasma half-life is extended or reduced. It is known that the binding of albumin to its receptor FcRn is important for plasma half-life and the correlation between receptor binding and plasma half-life is that a higher affinity of albumin to its receptor leads to longer plasma half-life. Thus for the invention a higher affinity of albumin to FcRn is considered indicative of an increased plasma half-life and a lower affinity of albumin to its receptor is considered indicative of a reduced plasma half-life.

In this application and claims the binding of albumin to its receptor FcRn is described using the term affinity (KD) and the expressions “stronger” or “weaker”. Thus, it should be understood that a molecule having a higher affinity to FcRn than HSA is considered to bind more strongly to FcRn than HSA and a molecule having a lower affinity to FcRn than HSA is considered to bind more weakly to FcRn than HSA. Stronger binding may be defined (relative to a suitable reference) as an albumin-FcRn interaction having a binding affinity (KD) that is less than the binding affinity of HSA-FcRn or ‘reference molecule’-FcRn, such as less than 0.9×KD for HSA or the reference molecule, more preferred less than 0.5×KD, less than 0.1×KD, less than 0.05×, 0.02×KD and most preferred less than 0.01×KD for HSA or the reference molecule. Weaker binding may be defined similarly, for example, more than 1.1×KD for HSA or the reference molecule, more preferred at least 5×KD, at least 10×KD, or at least 100× for HSA or the reference molecule. KD may be determined by any suitable method such as that defined herein. Binding affinity may be determined against any FcRn, particularly human FcRn for example shFcRn such as GST-tagged FcRn as described herein.

The terms “longer plasma half-life” or “shorter plasma half-life” and similar expressions are understood to be in relationship to the corresponding parent (or reference) albumin molecule. Thus, a longer plasma half-life with respect to a variant albumin of the invention means that the variant has longer plasma half-life than the corresponding albumin, i.e. the first (parent) albumin.

A ‘long’ or ‘short’ plasma half-life of, for example, a polypeptide, variant, fusion, conjugate or associate, for example in blood, is relative to a ‘reference molecule’. The reference molecule may be selected from: (i) a wild-type albumin (or a fragment thereof), (ii) an albumin variant (or fragment thereof), (iii) albumin (or a fragment thereof) fused to a polypeptide of interest, (iv) a conjugate of an albumin (or a fragment thereof), (v) an associate of an albumin (or a fragment thereof). More specifically, the ‘albumin’ of the reference molecule is preferably the first (parent) albumin as defined herein.

For example, long plasma half-life may be at least 5% longer than that of a reference molecule, preferably at least 10%, 20%, 30%, 40%, 50%, 60%, 70%, 80%, 90%, 100%, 150%, 200%, 250%, 300%, 350%, 400%, 450%, 500% longer. Long plasma half-life includes from at least to 100 days, for example at least 5, 6, 7, 8, 9, 10, 14, 15, 20, 21, 28, 30, 35, 40, 42, 50, 60, 70, 80, 90, 100 days. Short plasma half-life may be at least 5% shorter than that of a reference molecule, more preferably at least 10%, 20%, 30%, 40%, 50%, 60%, 70%, 80%, 90%, 95%, 96%, 97%, 98%, or 99% shorter. Short plasma half-life includes at most 5, 4, 3, 2, 1, 0.5, or 0.25 days.

The binding of an albumin variant to FcRn may also be described using kinetic factors, in particular “on-rate” (ka) and “off-rate” (kd), which describes the reaction rate whereby the albumin variant of the invention associated or dissociated with FcRn respectively. The inventors have further realized that the kinetics whereby the albumin variant interacts with FcRn may have an impact on the plasma half-life, and have realized that an albumin variant having a slow off-rate has a higher plasma half-life than a comparable molecule having a faster off-rate.

The correlation between binding of the albumin variant to the FcRn receptor and plasma half-life has been realized by the inventors based on the prior art within the field of this invention.

One way to determine whether the affinity of the albumin variant is higher or lower than wild-type albumins is using the Surface Plasmon Resonance assay (SPR) as described below. The skilled person will understand that other method might be useful to determine whether the affinity of the albumin variant to FcRn is higher or lower than the affinity of the corresponding reference albumin to FcRn, e.g. determination and comparison of the binding constants KD. Thus, according to the invention the albumin variant having a KD that is lower than the KD for natural HSA is considered to have a higher plasma half-life than HSA and albumin variants having a KD that is higher than the KD for natural HSA is considered to have a lower plasma half-life than HSA.

Reference: a reference is an albumin, fusion, conjugate, composition, associate, nanoparticle or microparticle to which an albumin variant, fusion, conjugate, composition, associate, nanoparticle or microparticle is compared. The reference may comprise or consist of full length albumin (such as HSA or a natural allele thereof) of a fragment thereof. A reference may also be referred to as a ‘corresponding’ albumin, fusion, conjugate, composition, associate nanoparticle or microparticle to which an albumin variant, fusion, conjugate, composition, associate nanoparticle or microparticle. A reference may comprise or consist of HSA (SEQ ID NO: 2) or a fragment, fusion, conjugate, associate, nanoparticle or microparticle thereof. Preferably, the reference is identical to the polypeptide, fusion polypeptide, conjugate, composition, associate, nanoparticle or microparticle according to the invention (“being studied”) with the exception of the albumin moiety. Preferably the albumin moiety of the reference comprises or consists of an albumin (e.g. HSA, SEQ ID NO: 2) or a fragment thereof. The amino acid sequence of the albumin moiety of the reference may be longer than, shorter than or, preferably, the same (±1 to 15 amino acids) length as the amino sequence of the albumin moiety of the polypeptide, fusion polypeptide, conjugate, composition, associate, nanoparticle or microparticle according to the invention (“being studied”).

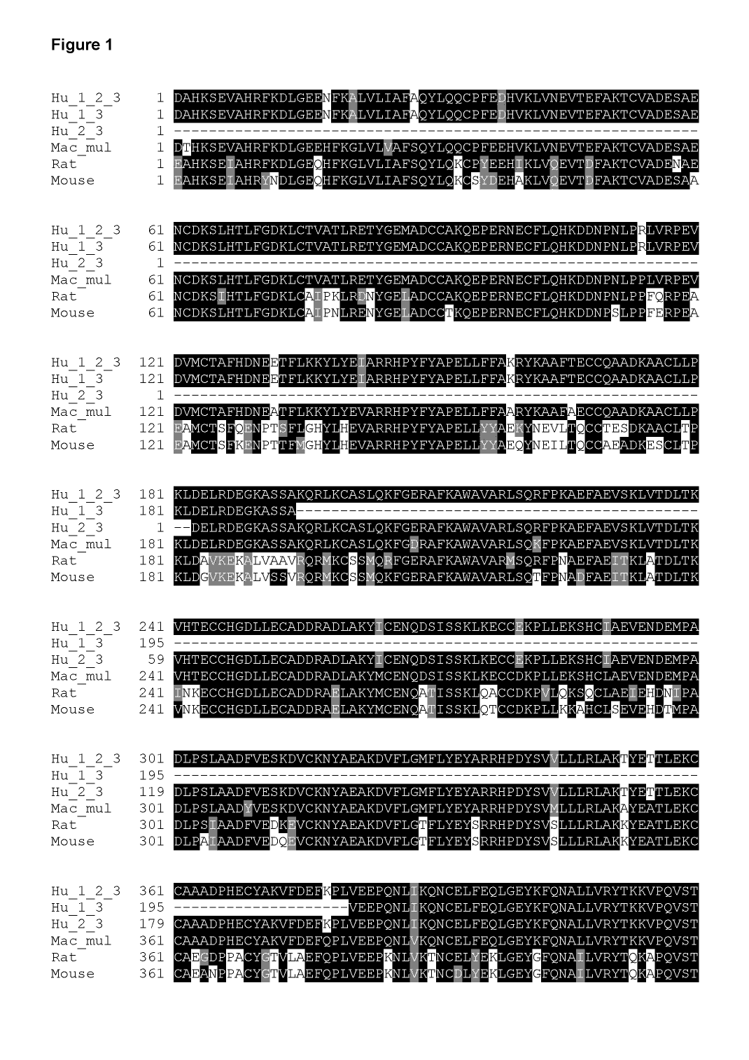

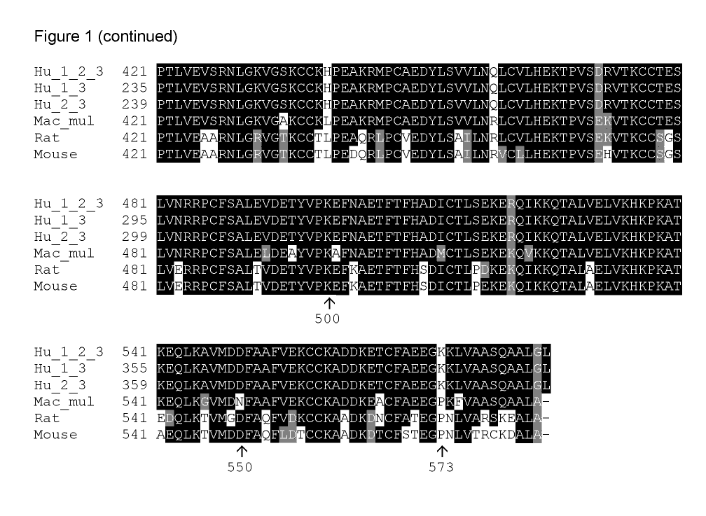

Equivalent amino acid positions: Throughout this specification amino acid positions are defined in relation to full-length mature human serum albumin (i.e. without leader sequence). However, the equivalent positions can be identified in fragments of human serum albumin, in animal albumins and in fragments, fusions and other derivative or variants thereof by comparing amino acid sequences using pairwise (e.g. ClustalW) or multiple (e.g. MUSCLE) alignments. For example, FIG. 1 shows that positions equivalent to 500, 550 and 573 in full length human serum albumin are easily identified in fragments of human serum albumin and in albumins of other species. Positions 500, 550 and 573 are indicated by arrows. Further details are provided in Table 1 below:

TABLE 1
Albumins from different animals showing positions equivalent
to 500, 550 and 573 of HSA.

| Organism | Albumin | | | Position equivalent to | | |
|---|---|---|---|---|---|---|
| (accession | Full length | | Total length | human serum albumin | | |
| number of | or | Fragment | of mature | (native amino acid): | | |
| protein) | fragment | details | protein | 500 (K) | 550 (D) | 573 (K) |
| Homo sapiens | Full length | — | 585 | 500 (K) | 550 (D) | 573 (K) |
| (AAA98797) | | | | | | |
| SEQ ID No: 2 | | | | | | |
| Homo sapiens | Fragment | DI, DIII | 399 | 314 (K) | 364 (D) | 387 (K) |
| SEQ ID No: 24 | | | | | | |
| Homo sapiens | Fragment | DII, DIII | 403 | 318 (K) | 368 (D) | 391 (K) |
| SEQ ID No: 25 | | | | | | |
| Macaca mulatta | Full length | — | 584 | 500 (K) | 550 (N) | 573 (P) |
| (NP_001182578) | | | | | | |
| Mature sequence | | | | | | |
| of SEQ ID No: 6 | | | | | | |
| Rattus norvegicus | Full length | — | 584 | 500 (K) | 550 (D) | 573 (P) |
| (AAH85359) | | | | | | |
| Mature sequence | | | | | | |
| of SEQ ID No: 10 | | | | | | |
| Mus musculus | Full length | — | 584 | 500 (K) | 550 (D) | 573 (P) |
| (AAH49971) | | | | | | |
| Mature sequence | | | | | | |
| of SEQ ID No: 9 | | | | | | |

FIG. 1 was generated by MUSCLE using the default parameters including output in ClustalW 1.81 format. The raw output data was shaded using BoxShade 3.21 (http://www.ch.embnet.org/software/BOX form.html) using Output Format: RTF_new; Font Size: 10; Consensus Line: no consensus line; Fraction of sequences (that must agree for shading): 0.5; Input sequence format: ALN. Therefore, throughout this specification amino acid positions defined in human serum albumin also apply to equivalent positions in fragments, derivatives or variants and fusions of human serum albumin, animals from other species and fragments and fusions thereof. Such equivalent positions may have (i) a different residue number in its native protein and/or (ii) a different native amino acid in its native protein.

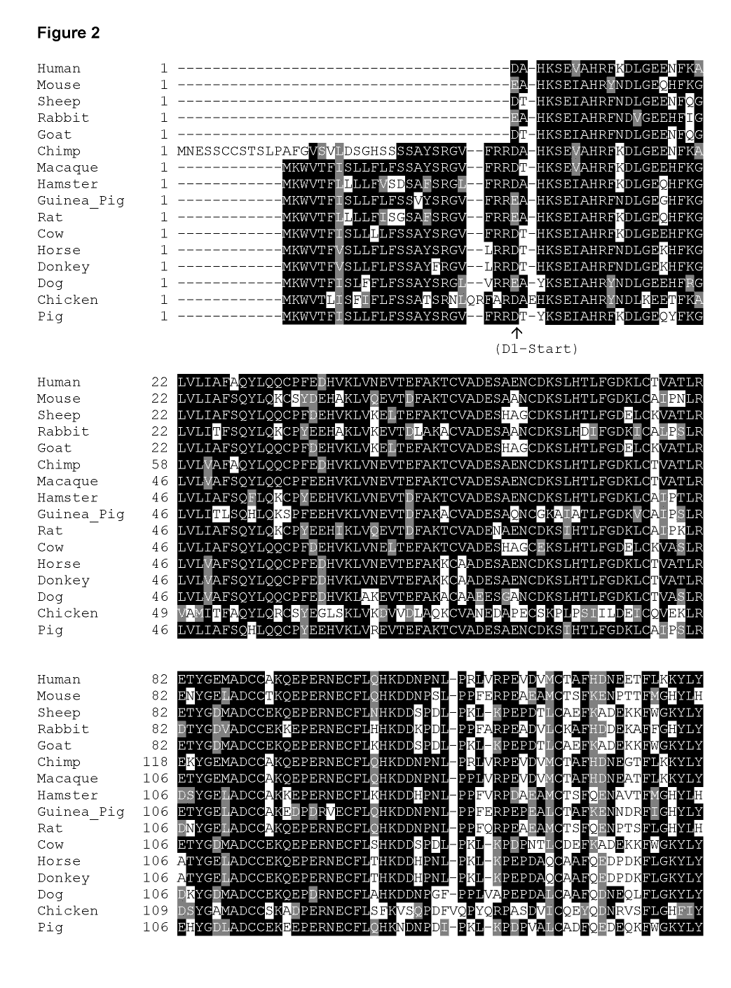

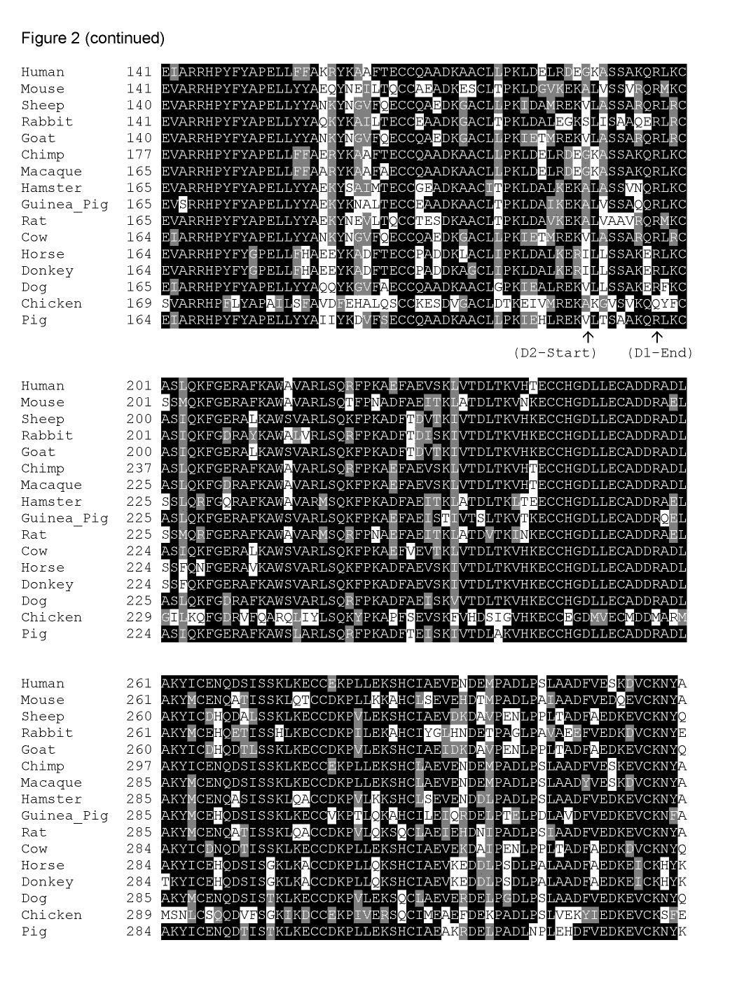

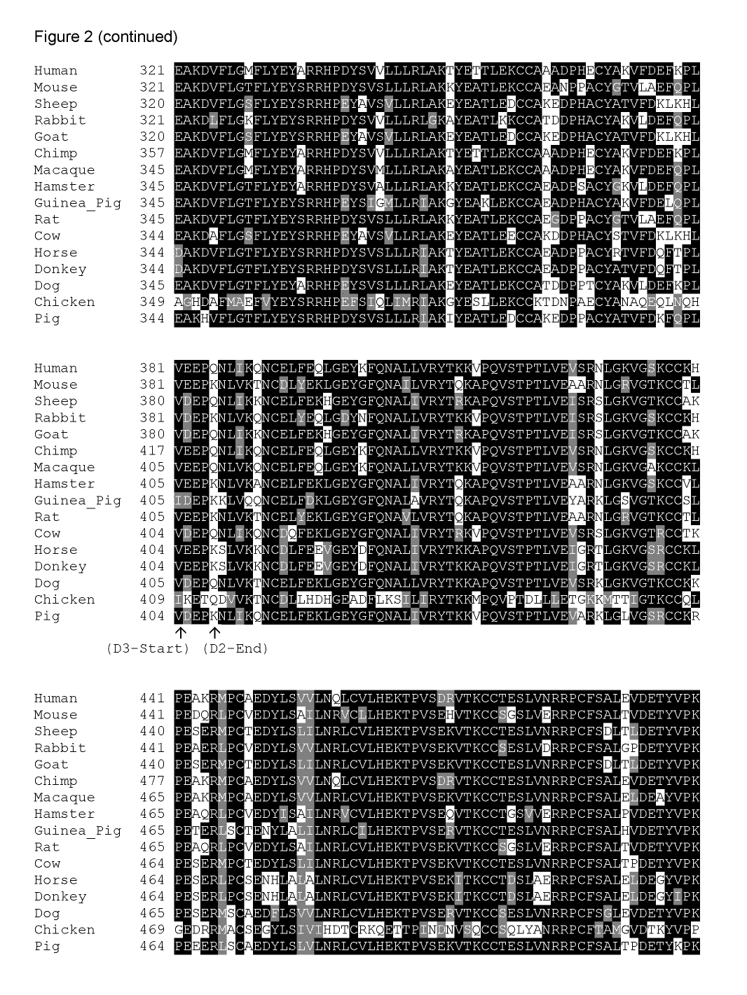

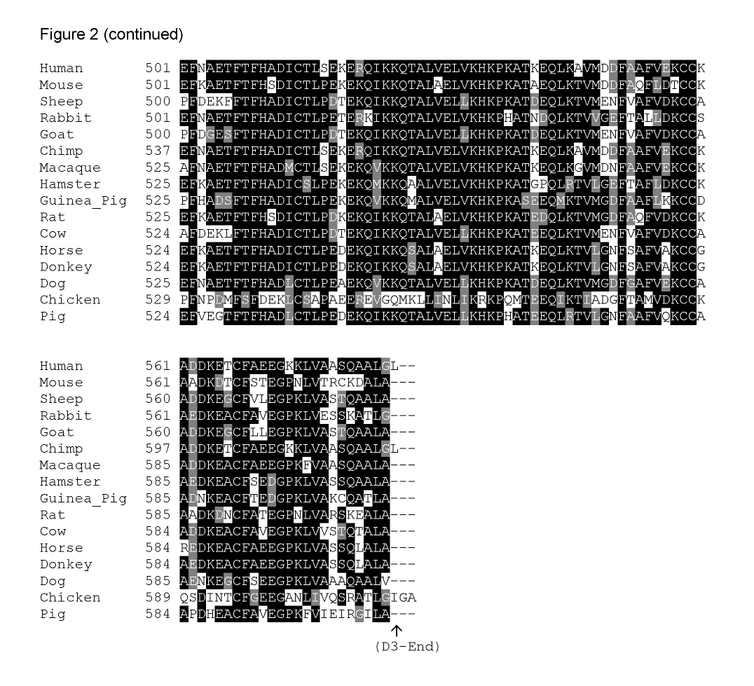

Likewise, FIG. 2 shows that equivalent positions can be identified in fragments (e.g. domains) of an albumin with reference to SEQ ID NO: 2 (HSA). FIG. 2 was generated by MUSCLE as described for FIG. 1, above.

### Conventions for Designation of Variants

For purposes of the invention, the mature polypeptide disclosed in SEQ ID NO: 2 is used to determine the corresponding amino acid residue in another albumin. The amino acid sequence of another albumin is aligned with the mature polypeptide disclosed in SEQ ID NO: 2, and based on the alignment, the amino acid position number corresponding to any amino acid residue in the mature polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later.

Identification of the corresponding amino acid residue in another albumin can be confirmed by an alignment of multiple polypeptide sequences using “ClustalW” (Larkin et al., 2007, Bioinformatics 23: 2947-2948).

When the other polypeptide (or protein) has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as inputs to a neural network that predicts the structural fold for a query sequence. Similarly, the method of Gough et al., 2000, J. Mol. Biol. 313: 903-919, can be used to align a sequence of unknown structure within the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementations of these algorithms can additionally be utilized to query structure databases with a structure of interest in order to discover possible structural homologs (e.g. Holm and Park, 2000, Bioinformatics 16: 566-567).

In describing the albumin variants of the invention, the nomenclature described below is adapted for ease of reference. The accepted IUPAC single letter or three letter amino acid abbreviation is employed. The term ‘point mutation’ and/or ‘alteration’ includes deletions, insertions and substitutions.

Substitutions.

For an amino acid substitution, the following nomenclature is used: Original amino acid, position, substituted amino acid. Accordingly, for example the substitution of threonine with alanine at position 226 is designated as “Thr226Ala” or “T226A”. Multiple mutations (alterations) are separated by addition marks (“+”), e.g. “Gly205Arg+Ser411Phe” or “G205R+S411F”, representing substitutions at positions 205 and 411 of glycine (G) with arginine (R) and serine (S) with phenylalanine (F), respectively.

Deletions.

For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g. “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple Alterations.

Variants comprising multiple alterations are separated by addition marks (“+”), e.g. “Arg170Tyr+Gly195Glu” or “R170Y+G195E” representing a substitution of tyrosine and glutamic acid for arginine and glycine at positions 170 and 195, respectively.

Different Substitutions.

Where different substitutions can be introduced at a position, the different substitutions are separated by a comma, e.g. “Arg170Tyr,Glu” represents a substitution of arginine with tyrosine or glutamic acid at position 170. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### Albumin Moiety

The albumin part of a fusion polypeptide, conjugate, associate, nanoparticle, microparticle or composition comprising the albumin variant or fragment thereof according to the invention, may be referred to as an ‘albumin moiety’. A polypeptide according to the invention may comprise or consist of an albumin moiety.

### Parent Albumin

Albumins are proteins and constitute the most abundant protein in plasma in mammals and albumins from a long number of mammals have been characterized by biochemical methods and/or by sequence information. Several albumins, e.g. human serum albumin (HSA), and horse albumin have also been characterized crystallographically and the structure determined (HSA: He XM, Carter DC (July 1992). “Atomic structure and chemistry of human serum albumin”. Nature 358 (6383): 209-15; horse albumin: Ho, J. X. et al. (2001). X-ray and primary structure of horse serum albumin (Equus caballus) at 0.27-nm resolution. Eur J. Biochem. 215(1):205-12).

HSA is a preferred first or second (parent) albumin according to the invention and is a protein consisting of 585 amino acid residues and has a molecular weight of 67 kDa. In its natural form it is not glycosylated. The amino acid sequence of HSA is shown in SEQ ID NO: 2. The skilled person will appreciate that natural alleles may exist having essentially the same properties as HSA but having one or more amino acid changes compared with SEQ ID NO: 2, and the inventors also contemplate the use of such natural alleles as parent albumin(s) according to the invention.

Albumins have generally a long plasma half-life of approximately 20 days or longer, e.g. HSA has a plasma half-life of 19 days. It is known that the long plasma half-life of HSA is mediated via interaction with its receptor FcRn, however, an understanding or knowledge of the exact mechanism behind the long half-life of HSA is not essential for the invention.

According to the invention the term “albumin” means a protein having the same, or very similar three dimensional structure as HSA and having a long plasma half-life. The term “albumin” also means a protein having the same and/or very similar three dimensional structure as HSA or HSA domains and has similar properties. Similar three dimensional structures are for example the structures of the albumins from the species mentioned under parent albumin. Some of the major properties of albumin are its ability to regulate of plasma volume since it contributes to 85% of the osmotic effect of normal plasma, a long plasma half-life of around 19 days±5 days, ligand-binding, e.g. binding of endogenous molecules such as acidic, lipophilic compounds including bilirubin fatty acids, hemin and thyoxine (see also Table 1 of Kragh-Hansen et al, 2002, Biol. Pharm. Bull. 25, 695, hereby incorporated by reference), binding of small organic compounds with acidic or electronegative features e.g. drugs such as warfarin, diazepam, ibuprofen and paclitaxel (see also Table 1 of Kragh-Hansen et al, 2002, Biol. Pharm. Bull. 25, 695, hereby incorporated by reference). Not all of these properties need to be fulfilled to in order to characterize a protein or fragment as an albumin. As examples of albumin proteins according to the invention can be mentioned human serum albumin (e.g. AAA98797 or P02768-1, SEQ ID NO: 2 (mature), SEQ ID NO: 4 (immature)), primate serum albumin, (such as chimpanzee serum albumin (e.g. predicted sequence XP_{—}517233.2 SEQ ID NO: 5), gorilla serum albumin or macaque serum albumin (e.g. NP_{—}001182578, SEQ ID NO: 6)), rodent serum albumin (such as hamster serum albumin (e.g. A6YF56, SEQ ID NO: 7), guinea pig serum albumin (e.g. Q6WDN9-1, SEQ ID NO: 8), mouse serum albumin (e.g. AAH49971 or P07724-1 Version 3, SEQ ID NO: 9) and rat serum albumin (e.g. AAH85359 or P02770-1 Version 2, SEQ ID NO: 10))), bovine serum albumin (e.g. cow serum albumin P02769-1, SEQ ID NO: 11), equine serum albumin such as horse serum albumin (e.g. P35747-1, SEQ ID NO: 12) or donkey serum albumin (e.g. Q5XLE4-1, SEQ ID NO: 13), rabbit serum albumin (e.g. P49065-1 Version 2, SEQ ID NO: 14), goat serum albumin (e.g. ACF10391, SEQ ID NO: 15), sheep serum albumin (e.g. P14639-1, SEQ ID NO: 16), dog serum albumin (e.g. P49822-1, SEQ ID NO: 17), chicken serum albumin (e.g. P19121-1 Version 2, SEQ ID NO: 18) and pig serum albumin (e.g. P08835-1 Version 2, SEQ ID NO: 19) or a polypeptide having at least 70, 75, 80, 85, 90, 95, 96, 97, 98 or at least 99% amino acid identity to such an albumin. Other examples of albumin, which are also included in the scope of this application, include ovalbumin (e.g. P01012.pro: chicken ovalbumin; 073860.pro: turkey ovalbumin). A mature albumin sequence can be identified from an immature albumin sequence using techniques known to the skilled person, for example alignment with HSA (for which the mature and immature regions are known). For example, immature HSA is 609 amino acids long in which amino acids 1 to 19 are a signal sequence (also known as a leader sequence or pre sequence), amino acids 20 to 24 are a pro sequence and amino acids 25 to 609 are the mature protein. The alignment in FIG. 2 allows the skilled person to predict mature sequences for several animal albumins (see “D1 Start”). HSA as disclosed in SEQ ID NO: 2 or any naturally occurring allele thereof, is the preferred albumin according to the invention.

The first (parent) albumin and/or second (parent) albumin, a fragment thereof, or albumin part of a fusion polypeptide comprising albumin or a fragment thereof according to the invention has generally a sequence identity to the sequence of a wild-type albumin (such as those described herein) preferably to the sequence of HSA shown in SEQ ID NO: 2 of at least 60%, preferably at least 70%, preferably at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, more preferred at least 96%, more preferred at least 97%, more preferred at least 98% and most preferred at least 99% or 100%. The sequence identity may be over the full-length of SEQ ID NO: 2 or over a molecule consisting or comprising of a fragment such as one or more domains of SEQ ID NO: 2 such as a molecule consisting of or comprising domain III (e.g. SEQ ID NO: 27), a molecule consisting of or comprising domain II and domain III (e.g. SEQ ID NO: 25), a molecule consisting of or comprising domain I and domain III (e.g. SEQ ID NO: 24), a molecule consisting of or comprising two copies of domain III. (e.g. SEQ ID NO: 26), a molecule consisting of or comprising three copies of domain III (e.g. SEQ ID NO: 28) or a molecule consisting of or comprising domain I and two copies of domain III (e.g. SEQ ID NO: 29).

The first or second (parent) albumin preferably comprises or consists of the amino acid sequence of SEQ ID NO: 4. The first or second (parent) albumin may comprise or consist of the mature polypeptide of SEQ ID NO: 2.

In another embodiment, the first or second (parent) albumin is an allelic variant of the mature polypeptide of SEQ ID NO: 2.

Amino acid sequences of albumins show the following identities to HSA (SEQ ID No: 2): chimpanzee (98.8%), macaque (93.3%), dog (80%), donkey (76.3%), horse (76.3%), hamster (76.2%), cow (75.8%), pig (75.1%), goat (74.8%), sheep (74.6%), rabbit (74.3%), rat (73.3%), mouse (72.3%), guinea pig (72.1%), chicken (47%). The identities were determined using the Needleman-Wunsch algorithm described above, using the amino acid sequences of FIG. 2. Human and chimpanzee albumin have a K at the position corresponding to 573 of HSA (SEQ ID No: 2), all other species analaysed above have a P at the position corresponding to 573 of HSA (SEQ ID No: 2). Therefore, in a preferred embodiment:

(i) The first albumin has at least 94% amino acid sequence identity to HSA (SEQ ID No: 2), such as at least 94, 95, 96, 97, 98, 99, 99.5%, more preferably at least 98% identity. The first albumin may be HSA (SEQ ID No: 2) or chimpanzee albumin (the mature sequence of SEQ ID No: 5). It is preferred that the first albumin does not have a P residue at the position corresponding to 573 in HSA (SEQ ID No: 2). It is more preferred that the first albumin has a K residue at the position corresponding to 573 in HSA (SEQ ID No: 2); and

(ii) The second albumin has from 45 to 98% amino acid sequence identity to HSA (SEQ ID No: 2), such as 45 to 70, 71, 72, 73, 74, 75, 76, 77, 78, 79, 80, 85, 90, 91, 92, 93, 94, 95, 96, 97, 98%, more preferably from 70 to 94%. It is preferred that the second albumin does not have a K residue at the position corresponding to 573 in HSA (SEQ ID No: 2). It is more preferred that the second albumin has a P residue at the position corresponding to 573 in HSA (SEQ ID No: 2).

It is particularly preferred that the first albumin has at least 94% amino acid sequence identity to HSA (SEQ ID No: 2) and the second albumin has less than 94% amino acid sequence identity to HSA (SEQ ID No: 2), even more preferred that the first albumin has at least 98% amino acid sequence identity to HSA (SEQ ID No: 2) and the second albumin has less than 94% amino acid sequence identity to HSA (SEQ ID No: 2).

The first and/or second (parent) albumin may be encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the mature polypeptide coding sequence of SEQ ID NO: 1, or (iii) the full-length complementary strand of (i) or (ii) (J. Sambrook, E. F. Fritsch, and T. Maniatis, 1989, Molecular Cloning, A Laboratory Manual, 2d edition, Cold Spring Harbor, N.Y.).

The polynucleotide of SEQ ID NO: 1 or a subsequence thereof, as well as the amino acid sequence of SEQ ID NO: 2 or a fragment thereof, may be used to design nucleic acid probes to identify and clone DNA encoding a parent from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g. at least 25, at least 35, or at least 70 nucleotides in length. Preferably, the nucleic acid probe is at least 100 nucleotides in length, e.g. at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labelled for detecting the corresponding gene (for example, with ³²P, ³H, ³⁵S, biotin, or avidin). Such probes are encompassed by the invention.

A genomic DNA or cDNA library prepared from such other organisms may be screened for DNA that hybridizes with the probes described above and encodes a parent. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1 or a subsequence thereof, the carrier material is used in a Southern blot.

For purposes of the invention, hybridization indicates that the polynucleotide hybridizes to a labelled nucleotide probe corresponding to the polynucleotide shown in SEQ ID NO: 1, its complementary strand, or a subsequence thereof, under low to very high stringency conditions. Molecules to which the probe hybridizes can be detected using, for example, X-ray film or any other detection means known in the art.

The nucleic acid probe may comprise or consist of the mature polypeptide coding sequence of SEQ ID NO: 1, i.e. nucleotides 1 to 1785 of SEQ ID NO: 1. The nucleic acid probe may comprise or consist of is a polynucleotide that encodes the polypeptide of SEQ ID NO: 2 or a fragment thereof. For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), 50° C. (low stringency), 55° C. (medium stringency), 60° C. (medium-high stringency), 65° C. (high stringency), or 70° C. (very high stringency).

For short probes that are about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

The parent may be encoded by a polynucleotide with a sequence identity to the mature polypeptide coding sequence of SEQ ID NO: 1 of at least 60%, e.g. at least 65%, at least 70%, at least 75%, at least 80%, at least 85%, at least 90%, at least 95%, at least 96%, at least 97%, at least 98%, at least 99%, or 100%, which encodes a polypeptide which is able to function as an albumin. In an embodiment, the parent is encoded by a polynucleotide comprising or consisting of SEQ ID NO: 1.

Particular aspects of the invention are discussed below:

A first aspect of the invention relates to polypeptides, which comprise (i) an N-terminal region of a first (parent) albumin, albumin variant or fragment thereof; and (ii) a C-terminal region of a second (parent) albumin, albumin variant or fragment thereof in which: (a) the N-terminal of the first (parent) albumin, albumin variant or fragment thereof comprises the amino acids of the molecule from which it is derived except the C-terminal 1 to 205 amino acids; and (b) the C-terminal of the second (parent) albumin, albumin variant or fragment thereof comprises the C-terminal 1 to 205 amino acids; and (c) the polypeptide has an altered half-life compared with the first (parent) albumin, albumin variant or fragment thereof and/or an altered binding affinity to FcRn compared with the first albumin, albumin variant or fragment thereof. The first albumin is not identical to the second albumin. Throughout this specification, the term ‘first (parent) albumin’ may be interchanged with ‘first albumin species’ and the term ‘second (parent) albumin’ may be interchanged with ‘second albumin species’. In this regard, the term species may be used in a taxonomic sense or in a non taxonomic sense. Preferably the C-terminal region of the first albumin is a maximum of 205, 204, 203, 202, 201, 200, 175, 150, 125, 100, 70, 60, 55, 50, 45, 40, 35, 30, 25, 20, 19, 18, 17, 16, 15, 14, 13, 12, 11, 10, 9, 8, 7, 6, 5, 4, 3, 2 or 1 amino acids long. Therefore, the C-terminal region may be from 1, 2, 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 amino acids long to 1, 2, 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 amino acids long. More preferably, the C-terminal region of the first albumin is 1 to 100, 2 to 85, 2 to 30, 10 to 30 and most preferably 12 to 20 amino acids long such as 12 or 13 amino acids long. Preferably, the C-terminal region of the second albumin is from 1, 2, 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 amino acids long to 1, 2, 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 amino acids long. More preferably, the C-terminal region of the second albumin is 1 to 100, 2 to 85, 2 to 30, 10 to 30, 2 to 20, and most preferably 12 or 13 amino acids long.

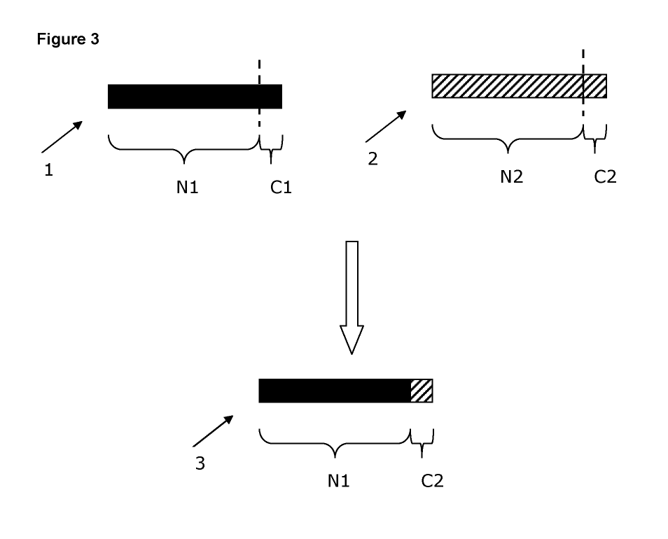

FIG. 3 is a schematic diagram of the first aspect of the invention. The first (1) and second (2) albumins, variants or fragments thereof are shown. A polypeptide according to the invention, i.e. a ‘chimeric’ albumin, (3) comprising the N-terminal region (N1) of the first albumin, variant or fragment thereof and the C-terminal region (C2) of the second albumin, variant or fragment thereof is also shown. In line with the invention disclosed herein, the first albumin may be considered to be a ‘recipient’ albumin and the second albumin may be considered to be a ‘donor’ albumin.

This first aspect of the invention may also be defined as a polypeptide which comprises (i) an N-terminal region of a first (parent) albumin, albumin variant or fragment thereof; and (ii) a C-terminal region of a second (parent) albumin, albumin variant or fragment thereof in which: (a) the N-terminal of the first (parent) albumin, albumin variant or fragment thereof comprises at least 65 to 100% of the albumin, albumin variant or fragment from which it is derived; and (b) the C-terminal of the second (parent) albumin, albumin variant or fragment thereof comprises the C-terminal at least 0.1 to 35% of the second albumin (c) the polypeptide has an altered half-life compared with the first (parent) albumin, albumin variant or fragment thereof and/or an altered binding affinity to FcRn compared with the first albumin, albumin variant or fragment thereof.

Preferably the N-terminal region of the first albumin is at least 65, 65.1, 65.2, 65.3, 65.4, 65.5, 65.6, 65.7, 65.8, 65.9, 66, 67, 68, 69, 70, 80, 81, 82, 83, 84, 85, 86, 87, 88, 89, 90, 95, 96, 97, 98, 99, 99.5, 99.6, 99.7, 99.8, 99.9% or 100% of the length of the albumin from which it is derived. More preferably, the N-terminal region of the first albumin is 83 to 99.5%, 95 to 98% and most preferably 96.5 to 98% of the length of the albumin from which it is derived. Preferably the C-terminal region of the second albumin is at least 0.1, 0.2, 0.3, 0.4, 0.5, 1, 2, 3, 4, 5, 6, 7, 8, 9, 10, 11, 12, 13, 14 15, 16, 17, 18, 19, 20, 25, 30, 31, 32, 33, 34, 34.1, 34.2, 34.3, 34.4, 34.5, 34.6, 34.7, 34.8, 34.9 or 35% of the length of the albumin from which it is derived. More preferably, the C-terminal region of the second albumin is 0.1 to 17%, 0.5 to 17%, 2 to 5% and most preferably 2 to 3.5% of the length of the albumin from which it is derived

The second (parent) albumin may be located at the C-terminus of the first (parent) albumin, albumin variant or fragment thereof or at the N-terminus of the first (parent) albumin, albumin variant or fragment thereof. Preferably, the second (parent) albumin, albumin variant or fragment thereof is located at the C-terminus of the first (parent) albumin, albumin variant or fragment thereof.

The first and/or second albumin may be selected from any albumin such as human serum albumin (e.g. AAA98797 or P02768-1, SEQ ID NO: 2 (mature), SEQ ID NO: 4 (immature)), primate serum albumin, (such as chimpanzee serum albumin (e.g. predicted sequence XP_{—}517233.2 SEQ ID NO: 5), gorilla serum albumin or macaque serum albumin (e.g. NP_{—}001182578, SEQ ID NO: 6), rodent serum albumin (such as hamster serum albumin (e.g. A6YF56, SEQ ID NO: 7), guinea pig serum albumin (e.g. Q6WDN9-1, SEQ ID NO: 8), mouse serum albumin (e.g. AAH49971 or P07724-1 Version 3, SEQ ID NO: 9) and rat serum albumin (e.g. AAH85359 or P02770-1 Version 2, SEQ ID NO: 10))), bovine serum albumin (e.g. cow serum albumin P02769-1, SEQ ID NO: 11), equine serum albumin such as horse serum albumin (e.g. P35747-1, SEQ ID NO: 12) or donkey serum albumin (e.g. Q5XLE4-1, SEQ ID NO: 13), rabbit serum albumin (e.g. P49065-1 Version 2, SEQ ID NO: 14), goat serum albumin (e.g. ACF10391, SEQ ID NO: 15), sheep serum albumin (e.g. P14639-1, SEQ ID NO: 16), dog serum albumin (e.g. P49822-1, SEQ ID NO: 17), chicken serum albumin (e.g. P19121-1 Version 2, SEQ ID NO: 18) and pig serum albumin (e.g. P08835-1 Version 2, SEQ ID NO: 19) or a polypeptide having at least 70, 75, 80, 85, 90, 95, 96, 97, 98 or at least 99% amino acid identity to such an albumin. The first and/or second albumin may be in immature (i.e. with pre-sequence and/or pro-sequence) or in mature form. The first albumin is not identical to the second albumin.

Preferably, the polypeptide comprises all amino acids of the first albumin, albumin variant or fragment thereof with the exception of the C-terminal 1 to 205 amino acids such as the C terminal 2, 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 to 3, 4, 5, 6, 7, 8, 9, 10, 11, 12, 13, 14, 15, 16, 17, 18, 19, 20, 25, 30, 35, 40, 45, 50, 55, 60, 70, 100, 125, 150, 175, 200, 201, 202, 203, 204 or 205 amino acids amino acids. Preferably the polypeptide comprises at least 65, 65.1, 65.2, 65.3, 65.4, 65.5, 65.6, 65.7, 65.8, 65.9, 66, 67, 68, 69, 70, 80, 90, 95, 96, 97, 98, 99, 99.5, 99.6, 99.7, 99.8, 99.9 or 100% of the first albumin, albumin variant or fragment thereof. A fragment may comprise or consist of at least the N-terminal 65, 65.1, 65.2, 65.3, 65.4, 65.5, 65.6, 65.7, 65.8, 65.9, 66, 67, 68, 69, 70, 80, 85, 90, 95, 96, 97, 98 or 99% of a full-length albumin and/or comprise or consist of at least the N-terminal 20, 50, 100, 200, 300, 400, 450, 475, 500, 525, 550, 560, 565, 566, 567, 568, 569, 570, 571, 572, 573, 574, 575, 576, 577, 578, 579, 580, 581, 582, 583 or 584 amino acids of a full-length albumin, such as an albumin as disclosed above. The fragment may or may not comprise one or more (several) point mutation as mentioned below.

It is particularly preferred that the polypeptide according to the invention comprises a Pro residue at position 573 (numbered relative to wt HSA). The skilled person can identify positions equivalent to 573 in other albumins and/or in fragments by sequence alignment such as those described herein, particularly those provided in FIG. 1 and FIG. 2.

The polypeptide may be longer, the same length, or shorter than the first (parent) or second (parent) albumin (or variant or fragment thereof) from which it is derived. For example, the polypeptide may be from 180 to 700 amino acids long such as from 180, 200, 250, 300, 350, 400, 450, 500, 550, 600, 650, 700 to 180, 200, 250, 300, 350, 400, 450, 500, 550, 600, 650, 700 amino acids long. Preferably the polypeptide of the first aspect of the invention is the same length, plus or minus 1, 2, 3, 4, 5, 6, 7, 8, 9, 10, 11, 12, 13, 14 or 15 amino acids, as the first (parent) albumin from which it is derived, more preferably the same length of plus or minus 1 or 2 amino acids.

The first (parent) albumin may comprise or consist of (i) domain III of an albumin, (ii) domain I and domain III of one or more albumins, (iii) domain II and domain III of one or more albumins, or (iv) a fragment of one or more domains described in (i), (ii) or (iii). Such a fragment may be or may comprise the C-terminal at least 80, 85, 90, 91, 92, 93, 94, 95, 96, 97, 98 or 99% of a domain III and/or the C-terminal at least 165, 170, 175, 180, 185, 190, 192, 194, 196, 198, 200, 201, 202, 203, 204 or 205 amino acids of domain III. Domains I, II and III may be defined with reference to HSA (SEQ ID NO: 2). For example, HSA domain I may consist of or comprise amino acids 1 to 194 (±1 to 15 amino acids) of SEQ ID NO: 2, HSA domain II may consist of or comprise amino acids 192 (±1 to 15 amino acids) to 387 (±1 to 15 amino acids) of SEQ ID NO: 2 and domain III may consist of or comprise amino acid residues 381 (±1 to 15 amino acids) to 585 (±1 to 15 amino acids) of SEQ ID NO: 2. “±1 to 15 amino acids” means that the residue number may deviate by plus or minus 1, 2, 3, 4, 5, 6, 7, 8, 9, 10, 11, 12, 13, 14, or 15 amino acids to the C-terminus and/or to the N-terminus of the stated amino acid position. Examples of domains I, II and III are described by Dockal et al (The Journal of Biological Chemistry, 1999, Vol. 274(41): 29303-29310) and Kjeldsen et al (Protein Expression and Purification, 1998, Vol 13: 163-169) and are tabulated below.

| Amino acid residues of HSA domains I, | | |
|---|---|---|
| II and III with reference to SEQ ID NO: 2 | Dockal et al | Kjeldsen et al |
| Domain I | 1 to 197 | 1 to 192 |
| Domain II | 189 to 385 | 193 to 382 |
| Domain III | 381 to 585 | 383 to 585 |

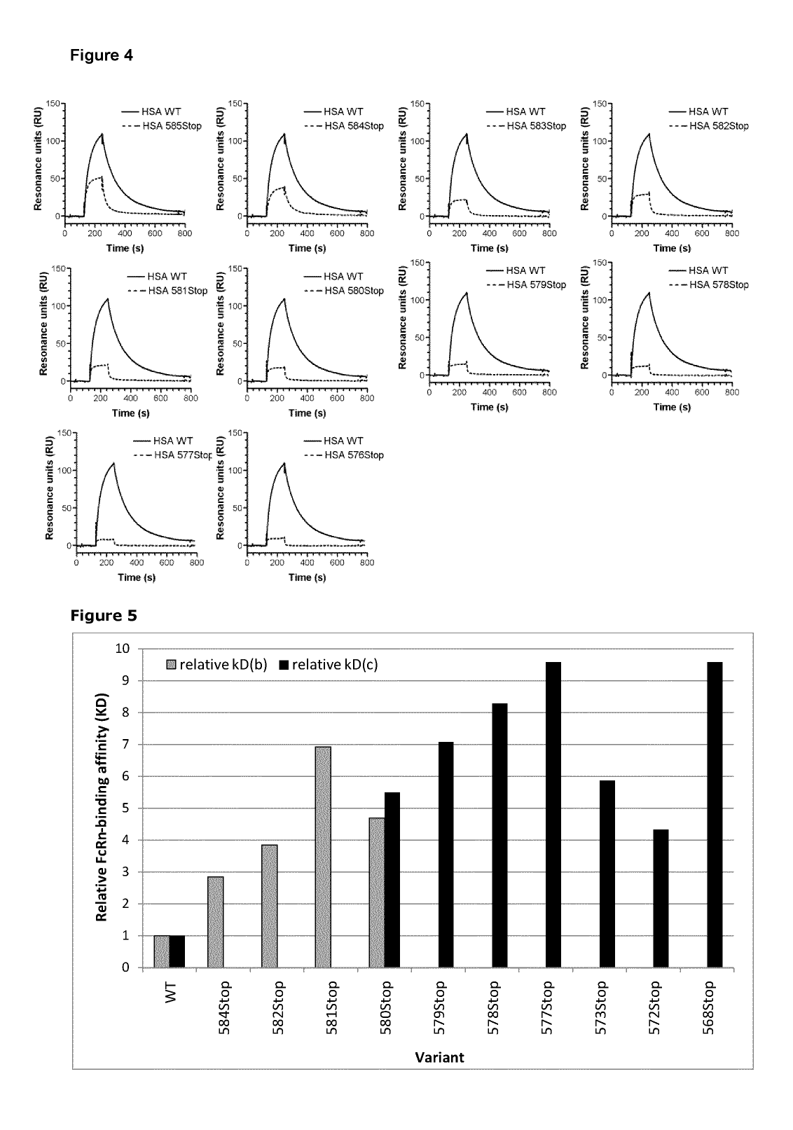

The skilled person can identify domains I, II and III in non-human albumins by amino acid sequence alignment with HSA, for example using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. Other suitable software includes MUSCLE ((Multiple sequence comparison by log-expectation, Robert C. Edgar, Version 3.6, http://www.drive5.com/muscle; Edgar (2004) Nucleic Acids Research 32(5), 1792-97 and Edgar (2004) BMC Bioinformatics, 5(1):113) which may be used with the default settings as described in the User Guide (Version 3.6, September 2005). Versions of MUSCLE later than 3.6 may also be used for any aspect of the invention). Examples of suitable alignments are provided in FIGS. 4 and 5 of PCT/EP2011/055577 (WO 2011/124718, the whole document is incorporated herein by reference).

The (chimeric) polypeptide of the invention may comprise the N-terminal region of a first albumin, albumin variant or fragment thereof; and (ii) a C-terminal region of a second albumin, albumin variant or fragment thereof as described herein in which when the amino acid sequences of the first albumin and the second albumin are aligned, the first amino acid of the C-terminal of the second albumin starts at the amino acid which is immediately downstream of the last amino acid of the N-terminal of the first albumin. For example, the polypeptide may comprise amino acids 1 to 572 of mature HSA C-terminus of a first albumin) and amino acids 573 to 584 of mature macaque albumin or amino acids 572 to 583 of mature sheep albumin (i.e. N-terminus of a second albumin). That is, based on alignment of the first and second albumins (e.g. FIG. 1 or 2), it is preferred that the cross-over between the N-terminal region of the first albumin and the C-terminal region of the second albumin is made at equivalent amino acid positions.

The first or second albumin, or variant or fragment thereof, may or may not comprise a point mutation at one or more (several) of the following positions: 417, 440, 464, 490, 492, 493, 494, 495, 496, 499, 500, 501, 503, 504, 505, 506, 510, 535, 536, 537, 538, 540, 541, 542, 550, 573, 574, 575, 577, 578, 579, 580, 581, 582 and 584. The positions are defined with reference to full length HSA (SEQ ID NO: 2). The skilled person can identify equivalent positions in full-length albumins from other species and/or in variants or fragments of albumin from any species, for example by using multiple alignment software such as those described above. Examples of suitable alignments are provided in FIGS. 1 and 2.

HSA as disclosed in SEQ ID NO: 2 or any naturally occurring allele thereof, is a preferred first albumin according to the invention. Furthermore, HSA (SEQ ID NO: 2) is a preferred reference against which ‘longer’ or ‘shorter’ half-life or binding affinity can be compared.

The first and/or second albumin may or may not comprise an albumin into which one or more conjugatable Cys residues have been introduced (‘thio-albumin’) such as those described in WO2010/092135 (incorporated herein by reference). ‘Conjugatable’ means that the Cys is available for conjugation, for example it is not disulphide bonded to another Cys residue within the albumin.

The polypeptide according to the first aspect of the invention may have (i) a shorter half-life compared with the first albumin, variant or fragment thereof or, more preferably, (ii) a longer half-life compared with the first albumin, variant or fragment thereof. The terms ‘longer’ and ‘shorter’ are defined above. The polypeptide according to the first aspect of the invention may have (i) a weaker binding affinity to FcRn compared with the first albumin, variant or fragment thereof or, more preferably, (ii) a stronger binding affinity to FcRn compared with the first albumin, variant or fragment thereof. The terms ‘stronger’ and ‘weaker’ are defined above.

A second aspect of the invention relates to polynucleotides encoding the first aspect of the invention. The polynucleotide may or may not be part of a vector, such as a plasmid. The polynucleotide may or may not be comprised in a host cell or host organism. Therefore, the invention also relates to isolated polynucleotides that encode any of the variants of the invention. Furthermore, the invention also relates to nucleic acid constructs comprising a polynucleotide encoding a variant of the invention operably linked to one or more (several) control sequences that direct the expression of the coding sequence in a suitable host cell under conditions compatible with the control sequences.

A polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, which is recognized by a host cell for expression of the polynucleotide. The promoter sequence contains transcriptional control sequences that mediate the expression of the variant. The promoter may be any nucleic acid sequence that shows transcriptional activity in the host cell including mutant, truncated, and hybrid promoters, and may be obtained from genes encoding extracellular or intracellular polypeptides either homologous or heterologous to the host cell.

In a yeast host, useful promoters are obtained from the genes for Saccharomyces cerevisiae enolase (ENO-1), Saccharomyces cerevisiae protease A (PRA1), Saccharomyces cerevisiae protease B (PRB1), Saccharomyces cerevisiae translation elongation factor (TEF1), Saccharomyces cerevisiae translation elongation factor (TEF2), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The skilled person knows useful promoters for use in rice and mammalian cells, such as CHO or HEK. For example, in a rice host, useful promoters are obtained from cauliflower mosaic virus 35S RNA gene (CaMV35S), maize alcohol dehydrogenase (Adh1) and alpha Amy3.

In a mammalian host cell, such as CHO or HEK, useful promoters are obtained from Cytomegalovirus (CMV) and CAG hybrid promoter (hybrid of CMV early enhancer element and chicken beta-actin promoter), Simian vacuolating virus 40 (SV40).

The control sequence may also be a suitable transcription terminator sequence, which is recognized by a host cell to terminate transcription. The terminator sequence is operably linked to the 3′-terminus of the polynucleotide encoding the variant. Any terminator that is functional in the host cell may be used.

Preferred terminators for yeast host cells are obtained from the genes for Saccharomyces cerevisiae enolase, Saccharomyces cerevisiae cytochrome C (CYC1), Saccharomyces cerevisiae alcohol dehydrogenase (ADH1) and Saccharomyces cerevisiae glyceraldehyde-3-phosphate dehydrogenase. Other useful terminators for yeast host cells are described by Romanos et al., 1992, supra. The skilled person knows useful terminators for use in rice and mammalian cells, such as CHO or HEK. For example, in a rice host, preferred terminators are obtained from Agrobacterium tumefaciens nopaline synthase (Nos) and cauliflower mosaic virus 35S RNA gene (CaMV35S).

The control sequence may also be a suitable leader sequence, a nontranslated region of an mRNA that is important for translation by the host cell. The leader sequence is operably linked to the 5′-terminus of the polynucleotide encoding the variant. Any leader sequence that is functional in the host cell may be used.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Useful polyadenylation sequences for yeast host cells are described by Guo and Sherman, 1995, Mol. Cellular. Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding region naturally linked in translation reading frame with the segment of the coding region that encodes the variant. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant. However, any signal peptide coding region that directs the expressed variant into the secretory pathway of a host cell may be used.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra. The skilled person knows useful signal peptides for use in rice and mammalian cells, such as CHO or HEK.

Where both signal peptide and propeptide regions are present at the N-terminus of a variant, the propeptide region is positioned next to the N-terminus of the variant and the signal peptide region is positioned next to the N-terminus of the propeptide region.

The nucleotide, whether or not within a vector such as a plasmid, may be comprised in a host cell such as those disclosed herein.

A third aspect of the invention relates to a method of producing a polypeptide according to the invention. The method is applicable to all aspects of the invention including albumins, variants, fragments and fusions thereof. As described below, the skilled person knows how to produce fusions, conjugates, associates and compositions of polypeptides and can therefore produce fusions, conjugates, associates and compositions of the albumins, variants, and fragments thereof. Such a method may comprise:

(a) Providing a nucleic acid encoding said albumin, variant, fragment and/or fusion thereof;

(b) Expressing the nucleic acid in a suitable host cell; and

(c) Recovering the albumin, variant, fragment and/or fusion thereof.

Such methods are known to the skilled person and are provided in references such as (J. Sambrook, E. F. Fritsch, and T. Maniatis, 1989, Molecular Cloning, A Laboratory Manual, 2nd edition, Cold Spring Harbor, N.Y.).

A method for preparing a variant of albumin, a fragment thereof or a fusion polypeptide comprising the variant or fragment may comprise:

i) providing a polynucleotide encoding an N-terminal region of a first (parent) albumin, albumin variant or fragment thereof and a C-terminal region of a second (parent) albumin, albumin variant or fragment thereof and wherein the polynucleotide optionally encodes one or more (several) fusion partner polypeptides at the N- and/or C-terminal of the resultant albumin; in which:

(a) the N-terminal of the first (parent) albumin, albumin variant or fragment thereof comprises the amino acids of the molecule from which it is derived except the C-terminal 1 to 205 amino acids; and (b) the C-terminal of the second (parent) albumin, albumin variant or fragment thereof comprises the C-terminal 1 to 205 amino acids; and (c) the polypeptide has an altered half-life compared with the first (parent) albumin, albumin variant or fragment thereof and/or an altered binding affinity to FcRn compared with the first albumin, albumin variant or fragment thereof;

and/or

(a) the N-terminal of the first (parent) albumin, albumin variant or fragment thereof comprises at least 65 to 100% of the albumin, albumin variant or fragment from which it is derived; and (b) the C-terminal of the second (parent) albumin, albumin variant or fragment thereof comprises the C-terminal at least 0.5 to 35% of the second albumin (c) the polypeptide has an altered half-life compared with the first (parent) albumin, albumin variant or fragment thereof or other reference such as HSA and/or an altered binding affinity to FcRn compared with the first (parent) albumin, albumin variant or fragment thereof or other reference such as HSA

ii) expressing the polynucleotide in a host cell; and

iii) recovering the resultant polypeptide or fusion polypeptide.

Preferably properties of the resultant polypeptide are according to the first and/or fourth aspect of the invention, for example the location of the N and C terminals relative to each other; the source and/or sequence of the parent albumin(s), the length of the resultant albumin.

The skilled person understands that the preferences for the first aspect of the invention, particularly the parameters of the C′ and/or N′ terminus of the first and/or second albumin also apply to the third aspect of the invention. The skilled person also understands that any aspect of the invention may be combined with one or more other aspects of the invention and/or one or more of the claims.

The invention also provides a method for altering the half-life of a molecule comprising:

(a) where the molecule is a polypeptide, fusing the molecule to a polypeptide according to the invention or conjugating the molecule to a polypeptide or fusion polypeptide according to the invention;

(b) where the molecule is not a polypeptide, conjugating the molecule to a polypeptide or fusion polypeptide according to the invention.

Examples of ‘molecule’ include those useful in therapy, prophylaxis (including those used in vaccines either as an active pharmaceutical ingredient or as an excipient), imaging and diagnosis, such as those described herein.

The nucleic acid may be comprised in a genetic construct where the modified nucleic acid is placed in operative connection with suitable regulatory genetic elements, such as promoter, terminator, activation sites, ribosome binding sites etc. The genetic construct may be introduced into a suitable host organism, culturing the transformed host organism under conditions leading to expression of the variant and recovering the variant. All these techniques are known in the art and it is within the skills of the average practitioner to design a suitable method for preparing a particular variant according to the invention.

The variant polypeptide of the invention may also be connected to a signal sequence in order to have the variant polypeptide secreted into the growth medium during culturing of the transformed host organism. It is generally advantageous to have the variant polypeptide secreted into the growth medium in order to ease recovery and purification.

Techniques for preparing variant polypeptides have also been disclosed in WO 2009019314 (included by reference) and these techniques may also be applied to the invention.

Albumins have been successfully expressed as recombinant proteins in a range of hosts including fungi (including but not limited to Aspergillus (WO06066595), Kluyveromyces (Fleer 1991, Bio/technology 9, 968-975), Pichia (Kobayashi 1998 Therapeutic Apheresis 2, 257-262) and Saccharomyces (Sleep 1990, Bio/technology 8, 42-46)), bacteria (Pandjaitab 2000, J. Allergy Clin. Immunol. 105, 279-285)), animals (Barash 1993, Transgenic Research 2, 266-276) and plants (including but not limited to potato and tobacco (Sijmons 1990, Bio/technology 8, 217 and Farran 2002, Transgenic Research 11, 337-346) and rice e.g. Oryza sativa) and mammalian cells such as CHO and HEK. The variant polypeptide of the invention is preferably produced recombinantly in a suitable host cell. In principle any host cell capable of producing a polypeptide in suitable amounts may be used and it is within the skills of the average practitioner to select a suitable host cell according to the invention. A preferred host organism is yeast, preferably selected among Saccharomycacae, more preferred Saccharomyces cerevisiae.

The variant polypeptides of the invention may be recovered and purified from the growth medium using a combination of known separation techniques such as filtration, centrifugation, chromatography, and affinity separation techniques etc. It is within the skills of the average practitioner to purify the variants of the invention using a particular combination of such known separation steps. As an example of purification techniques that may be applied to the variants of the invention can be mentioned the teaching of WO0044772.

The variant polypeptides of the invention may be used for delivering a therapeutically or prophylactically (including vaccines) beneficial compound to an animal or a human individual in need thereof. Such therapeutically or prophylactically beneficial compounds include, but are not limited, to labels and readily detectable compounds for use in diagnostics, such as various imaging techniques; pharmaceutical active compounds such as drugs, or specifically binding moieties such as antibodies. The variants of the invention may even be connected to two or more different therapeutically or prophylactically beneficial compounds, e.g. an antibody and a drug, which gives the combined molecule the ability to bind specifically to a desired target and thereby provide a high concentration of the connected drug at that particular target.

A fourth aspect of the invention relates to fusions of an albumin, variant or fragment according to the invention and to methods of producing and/or using such fusions.

A fusion polypeptide according to the invention comprises an albumin, variant or fragment thereof and a ‘fusion partner’. A ‘fusion’ is preferably a genetic fusion, e.g. a single open reading frame encodes both the albumin part of the fusion and the fusion partner. The fusion partner may in principle be any polypeptide, but generally it is preferred that the fusion partner is a polypeptide having therapeutic, prophylactic or diagnostic properties. Fusion polypeptides comprising albumin are known in the art. It has been found that such a fusion polypeptide comprising or consisting of albumin and a fusion partner polypeptide have a longer plasma half-life compared with the unfused fusion partner polypeptide alone. According to the invention it is possible to alter the plasma half-life of the fusion polypeptides according to the invention compared with the corresponding fusion polypeptides of the prior art. Further teaching regarding albumin fusions to fusion partner polypeptides and examples of suitable fusion partner polypeptides can be found in WO 01/79271A (particularly page 9 and/or Table 1), WO 03/59934A (particularly Table 1) and WO01/078480 (particularly Table 1) (each incorporated herein by reference in their entirety). The half-life of an albumin fusion according to the invention may be longer or shorter than the half-life of the fusion partner polypeptide alone. The half-life of an albumin fusion according to the invention may be longer or shorter than the half-life of the analogous/equivalent albumin fusion comprising or consisting of native HSA (instead of an albumin variant or derivative according to the invention) and the fusion partner. Preferably the fusion partner is not an albumin, variant or fragment thereof.

A fifth aspect of the invention relates to conjugates (conjugations) of an albumin, variant or fragment according to the invention and to methods of producing and/or using such conjugates. Therefore, the albumins, variants or fragments thereof according to the invention may be conjugated to a second molecule (conjugation partner') using techniques known within the art. Said second molecule may comprise a therapeutic, prophylactic (e.g. vaccine) or diagnostic moiety. In a particular embodiment the conjugate may be useful as a diagnostic tool such as in imaging; or the second molecule may be a therapeutic or prophylactic compound and in this embodiment the conjugate may be used for therapeutic or prophylactic purposes where the conjugate will have the therapeutic or prophylactic properties of the therapeutic or prophylactic compound as well as the long plasma half-life of the albumin. Conjugates of albumin and a therapeutic or prophylactic molecule are known in the art and it has been verified that such conjugates have long plasma half-life compared with the non-conjugated, free therapeutic or prophylactic molecule as such. According to the invention it is possible to alter the plasma half-life of the conjugate according to the invention compared with the corresponding conjugates of the prior art. ‘Alter’ includes both increasing the plasma half-life and decreasing the plasma half-life. Increasing the plasma half-life is preferred. The conjugates may conveniently be linked via a free thio group present on the surface of HSA (amino acid residue 34 of mature HSA) using well known chemistry.

In one particular preferred aspect the variant albumin or fragment thereof is conjugated to a beneficial therapeutic or prophylactic (e.g. vaccine) compound and the conjugate is used for treatment or prevention of a condition in a patient in need thereof, which condition is responsive to the particular selected therapeutic or prophylactic compound. Techniques for conjugating such a therapeutic or prophylactic compound to the variant albumin or fragment thereof are known in the art. WO 2009/019314 discloses examples of techniques suitable for conjugating a therapeutically or prophylactically useful compound to a polypeptide which techniques can also be applied to the invention. Further WO 2009/019314 discloses examples of compounds and moieties that may be conjugated to substituted transferrin and these examples may also be applied to the invention. The teaching of WO 2009/019314 is included herein by reference.

HSA contains in its natural form one free thiol group that conveniently may be used for conjugation. As a particular embodiment within this aspect the variant albumin or fragment thereof may comprise further modifications provided to generate additional free thiol groups on the surface. This has the benefit that the payload of the variant albumin or fragment thereof is increased so that more than one molecule of the therapeutic or prophylactic compound can be conjugated to each molecule of variant albumin or fragment thereof, or two or more different therapeutic or prophylactic compounds may be conjugated to each molecule of variant albumin or fragment thereof, e.g. a compound having targeting properties such as an antibody specific for example a tumour; and a cytotoxic drug conjugated to the variant albumin or fragment thereof thereby creating a highly specific drug against a tumour. Teaching of particular residues that may be modified to provide for further free thiol groups on the surface can be found in co-pending patent application WO 2010/092135, which is incorporated by reference. Preferably the conjugation partner is not an albumin, variant or fragment thereof.

A sixth aspect of the invention relates to associates of an albumin, variant or fragment according to the invention and to methods of producing and/or using such associates. In this connection the term “associate” is intended to mean a compound comprising a variant of albumin or a fragment thereof and another compound bound or associated to the variant albumin or fragment thereof by non-covalent binding. As an example of such an associate can be mentioned an associate consisting variant albumin and a lipid associated to albumin by a hydrophobic interaction. Such associates are known in the art and they may be prepared using well known techniques. As an example of a preferred associate, according to the invention, can be mentioned an associate comprising variant albumin and paclitaxel. Further examples of associates comprise a therapeutic, prophylatic (including vaccine), diagnostic, imaging or other beneficial moiety such as those described herein.

The half-life of an albumin associate according to the invention may be longer or shorter than the half-life of the ‘other compound’ alone. The half-life of an albumin associate according to the invention may be longer or shorter than the half-life of the analogous/equivalent albumin associate comprising or consisting of a reference albumin such as native HSA (instead of an albumin variant or derivative according to the invention) and the ‘other compound’. Methods for the preparation of associates are well-known to the skilled person, for example, formulation (by association) of HSA with Lipo-compounds is described in Hussain, R. and Siligardi, G. (2006) International Journal of Peptide Research and Therapeutics, Vol. 12, NO: 3, pp. 311-315.

For all aspects of the invention fusion partner polypeptides and/or conjugates may comprise one or more (several) of: 4-1BB ligand, 5-helix, A human C—C chemokine, A human L105 chemokine, A human L105 chemokine designated huL105_{—}3, A monokine induced by gamma-interferon (MIG), A partial CXCR4B protein, A platelet basic protein (PBP), α1-antitrypsin, ACRP-30 Homologue; Complement Component C1q C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase B, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C—C chemokine, MCP2, CCR5 variant, CCR7, CCR7, CD11a Mab, CD137; 4-1BB Receptor Protein, CD20 Mab, CD27, CD27L, CD30, CD30 ligand, CD33 immunotoxin, CD40, CD40L, CD52 Mab, Cerebus Protein, Chemokine Eotaxin, Chemokine hIL-8, Chemokine hMCP1, Chemokine hMCP1a, Chemokine hMCP1b, Chemokine hMCP2, Chemokine hMCP3, Chemokine hSDF1b, Chemokine MCP-4, chemokine TECK and TECK variant, Chemokine-like protein IL-8M1 Full-Length and Mature, Chemokine-like protein IL-8M10 Full-Length and Mature, Chemokine-like protein IL-8M3, Chemokine-like protein IL-8M8 Full-Length and Mature, Chemokine-like protein IL-8M9 Full-Length and Mature, Chemokine-like protein PF4-414 Full-Length and Mature, Chemokine-like protein PF4-426 Full-Length and Mature, Chemokine-like protein PF4-M2 Full-Length and Mature, Cholera vaccine, Chondromodulin-like protein, c-kit ligand; SCF; Mast cell growth factor; MGF; Fibrosarcoma-derived stem cell factor, CNTF and fragment thereof (such as CNTFAx15′(Axokine™)), coagulation factors in both pre and active forms, collagens, Complement C5 Mab, Connective tissue activating protein-III, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_{—}7, DIL-40, Dnase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; gelonin, HST-1; HBGF-4, FGF-5, FGF-6; Heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, fragment. myofibrillar protein Troponin I, FSH, Galactosidase, Galectin-4, G-CSF, GDF-1, Gene therapy, Glioma-derived growth factor, glucagon, glucagon-like peptides, Glucocerebrosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GRO-gamma), hAPO-4; TROY, hCG, Hepatitus B surface Antigen, Hepatitus B Vaccine, HER2Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, Human Act-2 protein, Human adipogenesis inhibitory factor, human B cell stimulating factor-2 receptor, Human beta-chemokine H1305 (MCP-2), Human C—C chemokine DGWCC, Human CC chemokine ELC protein, Human CC type chemokine interleukin C, Human CCC3 protein, Human CCF18 chemokine, Human CC-type chemokine protein designated SLC (secondary lymphoid chemokine), Human chemokine beta-8 short forms, Human chemokine C10, Human chemokine CC-2, Human chemokine CC-3, Human chemokine CCR-2, Human chemokine Ckbeta-7, Human chemokine ENA-78, Human chemokine eotaxin, Human chemokine GRO alpha, Human chemokine GROalpha, Human chemokine GRObeta, Human chemokine HCC-1, Human chemokine HCC-1, Human chemokine 1-309, Human chemokine IP-10, Human chemokine L105_{—}3, Human chemokine L105_{—}7, Human chemokine MIG, Human chemokine MIG-beta protein, Human chemokine MIP-1alpha, Human chemokine MIP1beta, Human chemokine MIP-3alpha, Human chemokine MIP-3beta, Human chemokine PF4, Human chemokine protein 331D5, Human chemokine protein 61164, Human chemokine receptor CXCR3, Human chemokine SDF1alpha, Human chemokine SDF1beta, Human chemokine ZSIG-35, Human Chr19Kine protein, Human CKbeta-9, Human CKbeta-9, Human CX3C 111 amino acid chemokine, Human DNAX interleukin-40, Human DVic-1 C—C chemokine, Human EDIRF 1 protein sequence, Human EDIRF 11 protein sequence, Human eosinocyte CC type chemokine eotaxin, Human eosinophil-expressed chemokine (EEC), Human fast twitch skeletal muscle troponin C, Human fast twitch skeletal muscle troponin I, Human fast twitch skeletal muscle Troponin subunit C, Human fast twitch skeletal muscle Troponin subunit I Protein, Human fast twitch skeletal muscle Troponin subunit T, Human fast twitch skeletal muscle troponin T, Human foetal spleen expressed chemokine, FSEC, Human GM-CSF receptor, Human gro-alpha chemokine, Human gro-beta chemokine, Human gro-gamma chemokine, Human IL-16 protein, Human IL-1RD10 protein sequence, Human IL-1RD9, Human IL-5 receptor alpha chain, Human IL-6 receptor, Human IL-8 receptor protein hIL8RA, Human IL-8 receptor protein hIL8RB, Human IL-9 receptor protein, Human IL-9 receptor protein variant #3, Human IL-9 receptor protein variant fragment, Human IL-9 receptor protein variant fragment#3, Human interleukin 1 delta, Human Interleukin 10, Human Interleukin 10, Human interleukin 18, Human interleukin 18 derivatives, Human interleukin-1 beta precursor, Human interleukin-1 beta precursor, Human interleukin-1 receptor accessory protein, Human interleukin-1 receptor antagonist beta, Human interleukin-1 type-3 receptor, Human Interleukin-10 (precursor), Human Interleukin-10 (precursor), Human interleukin-11 receptor, Human interleukin-12 40 kD subunit, Human interleukin-12 beta-1 receptor, Human interleukin-12 beta-2 receptor, Human Interleukin-12 p35 protein, Human Interleukin-12 p40 protein, Human interleukin-12 receptor, Human interleukin-13 alpha receptor, Human interleukin-13 beta receptor, Human interleukin-15, Human interleukin-15 receptor from clone P1, Human interleukin-17 receptor, Human interleukin-18 protein (IL-18), Human interleukin-3, human interleukin-3 receptor, Human interleukin-3 variant, Human interleukin-4 receptor, Human interleukin-5, Human interleukin-6, Human interleukin-7, Human interleukin-7, Human interleukin-8 (IL-8), Human intracellular IL-1 receptor antagonist, Human IP-10 and HIV-1 gp120 hypervariable region fusion protein, Human IP-10 and human Muc-1 core epitope (VNT) fusion protein, human liver and activation regulated chemokine (LARC), Human Lkn-1 Full-Length and Mature protein, Human mammary associated chemokine (MACK) protein Full-Length and Mature, Human mature chemokine Ckbeta-7, Human mature gro-alpha, Human mature gro-gamma polypeptide used to treat sepsis, Human MCP-3 and human Muc-1 core epitope (VNT) fusion protein, Human MI10 protein, Human MI1A protein, Human monocyte chemoattractant factor hMCP-1, Human monocyte chemoattractant factor hMCP-3, Human monocyte chemotactic proprotein (MCPP) sequence, Human neurotactin chemokine like domain, Human non-ELR CXC chemokine H174, Human non-ELR CXC chemokine IP10, Human non-ELR CXC chemokine Mig, Human PAI-1 mutants, Human protein with IL-16 activity, Human protein with IL-16 activity, Human secondary lymphoid chemokine (SLC), Human SISD protein, Human STCP-1, Human stromal cell-derived chemokine, SDF-1, Human T cell mixed lymphocyte reaction expressed chemokine (TMEC), Human thymus and activation regulated cytokine (TARC), Human thymus expressed, Human TNF-alpha, Human TNF-alpha, Human TNF-beta (LT-alpha), Human type CC chemokine eotaxin 3 protein sequence, Human type II interleukin-1 receptor, Human wild-type interleukin-4 (hIL-4) protein, Human ZCHEMO-8 protein, Humanized Anti-VEGF Antibodies, and fragments thereof, Humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-10, IL-1 mature, IL-10 receptor, IL-11, IL-11, IL-12 p40 subunit, IL-13, IL-14, IL-15, IL-15 receptor, IL-17, IL-17 receptor, II-17 receptor, II-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, IL-4 mutein Y124G, IL-4 mutein Y124X, IL-4 muteins, II-5 receptor, IL-6, 11-6 receptor, IL-7 receptor clone, IL-8 receptor, IL-9 mature protein variant (Met117 version), immunoglobulins or immunoglobulin-based molecules or fragment of either (e.g. a Small Modular ImmunoPharmaceutical™ (“SMIP”) or dAb, Fab′ fragments, F(ab′)2, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon alpha species and sub-species, interferon beta species and sub-species, interferon gamma species and sub-species), interferons (such as interferon alpha species and sub-species, interferon beta species and sub-species, interferon gamma species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL10, IL11 and IL2), interleukins (such as IL10, IL11 and IL2), Japanese encephalitis vaccine, Kalikrein Inhibitor, Keratinocyte growth factor, Kunitz domain protein (such as aprotinin, amyloid precursor protein and those described in WO 03/066824, with or without albumin fusions), Kunitz domain protein (such as aprotinin, amyloid precursor protein and those described in WO 03/066824, with or without albumin fusions), LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody, MP52, Mutant Interleukin 6 S176R, myofibrillar contractile protein Troponin I, Natriuretic Peptide, Nerve Growth Factor-beta, Nerve Growth Factor-beta2, Neuropilin-1, Neuropilin-2, Neurotactin, Neurotrophin-3, Neurotrophin-4, Neurotrophin-4-a, Neurotrophin-4-b, Neurotrophin-4-c, Neurotrophin-4-d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, platelet-derived endothelial cell growth factor (PD-ECGF), Platelet-Derived Growth Factor A chain, Platelet-Derived Growth Factor B chain, polypeptide used to treat sepsis, Preproapolipoprotein “milano” variant, Preproapolipoprotein “paris” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, restrictocin, Retroviral protease inhibitors, ricin, Rotavirus Vaccine, RSV Mab, saporin, sarcin, Secreted and Transmembrane polypeptides, Secreted and Transmembrane polypeptides, serum cholinesterase, serum protein (such as a blood clotting factor), Soluble BMP Receptor Kinase Protein-3, Soluble VEGF Receptor, Stem Cell Inhibitory Factor, Straphylococcus Vaccine, Stromal Derived Factor-1 alpha, Stromal Derived Factor-1 beta, Substance P (tachykinin), T1249 peptide, T20 peptide, T4 Endonuclease, TACI, Tam, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor beta, Troponin peptides, Truncated monocyte chemotactic protein 2 (6-76), Truncated monocyte chemotactic protein 2 (6-76), Truncated RANTES protein (3-68), tumour necrosis factor, Urate Oxidase, urokinase, Vasopressin (Neurophysin II), VEGF R-3; flt-4, VEGF Receptor; KDR; flk-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9, alternative antibody scaffolds e.g. anticalin(s), adnectin(s), fibrinogen fragment(s), nanobodies such as camelid nanobodies, infestin, and/or any of the molecules mentioned in WO01/79271 (particularly page 9 and/or Table 1), WO 2003/59934 (particularly Table 1), WO03/060071 (particularly Table 1) or WO01/079480 (particularly Table 1) (each incorporated herein by reference in their entirety).

Furthermore, conjugates may comprise one or more (several) of chemotherapy drugs such as: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, A, Abraxane, Accutane®, Actinomycin-D, Adriamycin®, Adrucil®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin diftitox, DepoCyt™, Dexamethasone, Dexamethasone acetate, Dexamethasone Sodium Phosphate, Dexasone, Dexrazoxane, DHAD, DIC, Diodex, Docetaxel, Doxil®, Doxorubicin, Doxorubicin liposomal, Droxia™, DTIC, DTIC-Dome®, Duralone®, Efudex®, Eligard™, Ellence™, Eloxatin™, Elspar®, Emcyt®, Epirubicin, Epoetin alfa, Erbitux™, Erlotinib, Erwinia L-asparaginase, Estramustine, Ethyol, Etopophos®, Etoposide, Etoposide Phosphate, Eulexin®, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar®, Gleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Tositumomab, Trastuzumab, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®; radiopharmaceuticals such as: Carbon-11, Carbon-14, Chromium-51, Cobalt-57, Cobalt-58, Erbium-169, Fluorine-18, Gallium-67, Gold-198, Indium-111, Indium-113m, Iodine-123, Iodine-125, Iodine-131, Iron-59, Krypton-81m, Nitrogen-13, Oxygen-15, Phosphorous-32, Rhenium-186, Rubidium-82, Samarium-153, Selenium-75, Strontium-89, Technetium-99m, Thallium-201, Tritium, Xenon-127, Xenon-133, Yttrium-90; imaging agents such as Gadolinium, magnetite, manganese, technetium, I125, I131, P32, T1201, Iopamidol, PET-FDG.

Further fusion partners, conjugation partners and/or molecules for inclusion in a nanoparticle, microparticle, associate or composition according to the invention include: acromegaly drugs e.g. somatuline, lanreotide, octreotide, Sandostatin; antithrombotics e.g. bivalirudin, Angiomax, dalteparin, Fragmin, enoxaparin, Lovenox, Drotrecogin alfa (e.g. Activated), Xigris, heparin; assisted reproductive therapy compounds e.g. choriogonadotropin, Ovidrel, follitropin, alpha/beta; enzymes e.g. hyaluronidase, Hylenex; diabetes drugs e.g. exenatide, Byetta, glucagon, insulin, liraglutide, GLP-1 agonists; compounds useful in diagnosis e.g. protirelin, Thyrel TRH Thypinone, secretin (e.g. synthetic human), Chirhostim, thyrotropin (e.g. alpha), Thyrogen' erythropoiesis drugs e.g. Darbepoetin alfa, Aranesp, Epoetin alfa, Epogen, Eprex, drugs for the treatment of genetic defects e.g. pegademase, drugs for the treatment of growth failure e.g. Adagen, mecasermin, rinfabate, drugs for the treatment of cystic fibrosis e.g. Dornase alfa, Pulmozyme, drugs for the treatment of metabolic disorders e.g. Agalsidase beta, Fabrazyme, alglucosidase alpha, Myozyme, Laronidase, Aldurazyme, drugs for the treatment of genital wart intralesional e.g. Interferon alfa-n3, Alferon N, drugs for the treatment of granulomatous disease e.g. Interferon gamma-1b, Actimmune; drugs for the treatment of growth failure e.g. pegvisomant, Somavert, somatropin, Genotropin, Nutropin, Humatrope, Serostim, Protropin; drugs for the treatment of heart failure e.g. nesiritide, Natrecor; drugs for the treatment of hemophilia e.g. a coagulation factor e.g. Factor VIII, Helixate FS, Kogenate FS, Factor IX, BeneFIX, Factor Vila, Novoseven, desmopressin, Stimate, DDAVP; hemopoetic drugs e.g. Filgrastim (G-CSF), Neupogen, Oprelvekin, Neumega, Pegfilgrastim, Neulasta, Sargramostim, Leukine; drugs for the treatment of hepatitis C e.g. Interferon alfa-2a, Roferon A, Interferon alfa-2b, Intron A, Interferon alfacon-1, Infergen, Peginterferon alfa-2a, Pegasys, Peginterferon alfa-2b, PEG-Intron; drugs for the treatment of HIV e.g. enfuvirtide, Fuzeon; Fabs e.g. Fab (antithrombin), Abciximab, ReoPro; monoclonal antibodies e.g. Daclizumab, Zenapax; antiviral monoclonal antibodies e.g. Palivizumab, Synagis; monoclonal antibodies for the treatment of asthma e.g. Omalizumab, Xolair; monoclonal antibodies for use in diagnostic imaging e.g. Arcitumomab, CEA-Scan, Capromab Pendetide, ProstaScint, Satumomab Pendetide, OncoScint CR/OV, Fabs for use in diagnostic imaging e.g. Nofetumomab, Verluma; iimmuno-supressant monoclonal antibodies e.g. Basiliximab, Simulect, Muromonab-CD3, Orthoclone OKT3; monoclonal antibodies for the treatment of malignancy e.g. Alemtuzumab, Campath, Ibritumomab tiuxetan, Zevalin, Rituximab, Rituxan, Trastuzumab, Herceptin; monoclonal antibodies for the treatment of rheumatoid arthritis (RA) e.g. Adalimumab, Humira, Infliximab, Remicade; monoclonal antibodies for use as a radio-immuno-therapeutic e.g. Tositumomab and Iodine I¹³¹, Tositumomab, Bexxar; drugs for the treatment of macular degeneration e.g. pegaptanib, Macugen; drugs for the treatment of malignancy e.g. Aldesleukin, Proleukin, Interleukin-2, Asparaginase, Elspar, Rasburicase, Elitek, Denileukin diftitox, Ontak, Pegaspargase, Oncaspar, goserelin, leuprolide; drugs for the treatment of multiple sclerosis (MS) e.g. Glatiramer acetate (e.g. copolymer-1), Copaxone, Interferon beta-1a, Avonex, Interferon beta-1a, Rebif, Interferon beta-1b, Betaseron; drugs for the treatment of mucositis e.g. palifermin, Kepivance; drug for the treatment of dystonia e.g. neurotoxin, Botulinum Toxin Type A, BOTOX, BOTOX Cosmetic, Botulinum Toxin Type B, MYOBLOC; drugs for the treatment of osteoporosis e.g. teriparatide, Forteo; drugs for the treatment of psoriasis e.g. Alefacept, Amevive; drugs for the treatment of RA e.g. abatacept, Orencia, Anakinra, Kineret, Etanercept, Enbrel; thrombolytics e.g. Alteplase, Activase, rtPA, Anistreplase, Eminase, Reteplase, Retavase, Streptokinase, Streptase, Tenecteplase, TNKase, Urokinase, Abbokinase, Kinlytic; drugs for the treatment of osteoporosis e.g. calcitonin (e.g. salmon), Miacalcin, Fortical, drugs for the treatment of skin ulcers e.g. Becaplermin, Regranex, Collagenase, Santyl.

Such polypeptides and chemical compounds may be referred to as diagnostic moieties, therapeutic moieties, prophylactic moieties or beneficial moieties.

One or more (several) therapeutic or prophylactic polypeptides may be fused to the N-terminus, the C-terminus of albumin, inserted into a loop in the albumin structure or any combination thereof. It may or it may not comprise linker sequences separating the various components of the fusion polypeptide.

Teachings relating to fusions of albumin or a fragment thereof are known in the art and the skilled person will appreciate that such teachings can also be applied to the invention. WO 2001/79271A and WO 2003/59934A (incorporated herein by reference) also contain examples of therapeutic and prophylactic polypeptides that may be fused to albumin or fragments thereof, and these examples apply also to the invention.

A seventh aspect of the invention relates to compositions comprising an albumin, variant, fragment, fusion, conjugate or associate thereof as described herein and to the preparation of such compositions. The compositions are preferably pharmaceutical compositions. Therefore, the composition preferably comprises an albumin, variant, fragment, fusion, conjugate or associate thereof and a pharmaceutically acceptable carrier. The composition may be prepared using techniques known in the area such as disclosed in recognized handbooks within the pharmaceutical field. Since the albumin, variant, fragment, fusion, conjugate or associate thereof has a plasma-half life which is modulated (i.e. longer or shorter) than that of a reference molecule (defined above), the composition also has a modulated plasma-half life relative to an equivalent composition comprising the reference molecule in place of the albumin, variant, fragment, fusion, conjugate or associate thereof as described herein. The composition may be a vaccine. The polypeptide according to the invention may be an active pharmaceutical or an excipient. Optionally, the composition is provided in unit dosage form.

Preferably the albumin, variant, fragment, fusion, conjugate or associate thereof has a plasma half-life that is longer than the plasma half-life of the reference molecule e.g. the same composition except that the albumin component (e.g. albumin, variant, fragment, fusion, conjugate or associate) is wild-type albumin (e.g. HSA) or a variant, fragment, fusion, conjugate or associate.

Alternatively, this may be expressed as the albumin, variant, fragment, fusion, conjugate or associate thereof having a binding affinity (KD) to FcRn that is lower that the corresponding KD for HSA or the reference molecule. Preferably, KD for the albumin, variant, fragment, fusion, conjugate or associate thereof is less than 0.9×KD for HSA or the reference molecule, more preferred less than 0.5×KD, less than 0.1×KD, less than 0.05×, 0.02×KD and most preferred less than 0.01×KD for HSA or the reference molecule. The KD of the variant of albumin or a fragment thereof or fusion polypeptides comprising variant albumin or fragments thereof, fragment thereof, conjugate, nanoparticle, microparticle, associate or composition may be between the KD of wt albumin (e.g. SEQ ID No. 2) for FcRn and the KD of HSA K573P (SEQ ID No. 3) for FcRn.

The albumin, variant, fragment, fusion, conjugate or associate thereof is preferably according to the invention.

In a particular embodiment the composition comprises an albumin, variant, fragment, fusion, conjugate or associate thereof according to the invention and a compound comprising a pharmaceutically beneficial moiety and an albumin binding domain (ABD). According to the invention ABD means a site, moiety or domain capable of binding to circulating albumin in vivo and thereby conferring transport in the circulation of the ABD and any compound or moiety bound to said ABD. ABD's are known in the art and have been shown to bind very tight to albumin so a compound comprising an ABD bound to albumin will to a certain extent behave as a single molecule. The inventors have realized by using the albumin, variant, fragment, fusion, conjugate or associate thereof according to the invention together with a compound comprising a pharmaceutically beneficial moiety and an ABD makes it possible to alter the plasma half-life of the compound comprising a pharmaceutically beneficial moiety and an ABD compared with the situation where said compound were injected as such in a patient having need thereof or administered in a formulation comprising natural albumin or a fragment thereof.

The albumin, variant, fragment, fusion, conjugate or associate thereof may also be incorporated into nano- or microparticles using techniques well known within the art. Preferred methods for preparing nano- or microparticles that may be applied to the albumin, variant, fragment, fusion, conjugate or associate thereof according to the invention is disclosed in WO 2004/071536 or WO2008/007146 or Oner & Groves (Pharmaceutical Research, Vol 10(9), 1993, pages 1387 to 1388) which are incorporated herein by reference. Preferably the average diameter of a nano-particle is from 5 to 1000 nm, more preferably 5, 10, 20, 30, 40, 50, 80, 100, 130, 150, 200, 300, 400, 500, 600, 700, 800, 900, or 999 to 5, 10, 20, 30, 40, 50, 80, 100, 130, 150, 200, 300, 400, 500, 600, 700, 800, 900, or 1000 nm. An advantage of a microparticle less than 200 nm diameter, and more particularly less than 130 nm, is that is amenable to sterilisation by filtration through a 0.2 μm (micron) filter. Preferably, the average diameter of a microparticle is from 1000 nm (1 μm (micron)) to 100 μm (micron), more preferably from 1, 2, 5, 10, 20, 30, 40, 50, 60, 70, 80, 90, 100 to 1, 2, 5, 10, 20, 30, 40, 50, 60, 70, 80, 90, 100 μm (micron).

An eighth aspect of the invention relates to the use of or a method using an albumin, variant, fragment, fusion, conjugate or associate thereof as described herein in imaging. For example for a conjugate, associate or fusion polypeptide used for imaging purposes in animals or human beings, where the imaging moiety has an very short half-life and a conjugate or a fusion polypeptide comprising HSA has a plasma half-life that is far longer than needed for the imaging purposes it would be advantageous to use a variant albumin or fragment thereof of the invention having a shorter plasma half-life than the parent albumin or fragment thereof or other reference such as HSA, to provide conjugates of fusion polypeptides having a plasma half-life that is sufficiently long for the imaging purpose but sufficiently short to be cleared form the body of the particular patient on which it is applied.

A ninth aspect of the invention relates to method of treatment or prophylaxis or diagnosis including an albumin, variant, fragment, fusion, conjugate or associate or composition thereof as defined herein. In some situations, it would be advantageous to use an albumin, variant, fragment, fusion, conjugate or associate or composition thereof having a longer plasma half-life than the reference molecule or composition since this would have the benefit that the administration of the albumin, variant, fragment, fusion, conjugate or associate or composition thereof would be needed less frequently or at a reduced dose (and consequently with fewer side effects) compared with the situation where the reference molecule or composition was used.

A tenth aspect of the invention relates to use of a variant of albumin, fragment thereof or fusion polypeptide comprising said variant albumin or fragment thereof, conjugate thereof, associate thereof or composition thereof disclosed herein to alter the half-life, preferably in plasma, of a therapeutic, prophylatic, diagnostic, imaging or other beneficial moiety.

One advantage of the invention is that it allows the half-life of albumin, a variant of albumin or a fragment thereof or fusion polypeptides comprising variant albumin or fragments thereof, fragment thereof, conjugate, nanoparticle, microparticle, associate or composition to be tailored in order to achieve a binding affinity or half-life which meets the needs of the user. The invention is particularly applicable to pharmaceuticals. Some pharmaceuticals benefit from a long half-life, e.g. to increase dosage intervals. Some pharmaceuticals benefit from a short plasma half-life, e.g. to accelerate clearance from the body of a patient. Therefore, use of an albumin moiety according to the invention in pharmaceuticals allows the half-life of the pharmaceutical to be tailored as desired.

The skilled person understands that any aspect of the invention may be combined with another aspect or aspects of the invention and/or with one or more of the preferences for the aspects of the invention and/or other disclosures made herein.

The invention is further described with reference to the following examples that should not be construed as limiting the scope of the invention.

### EXAMPLES

The following methods were used to prepare and analyse albumin variants according to the invention:

### Method 1: Preparation of Variants of Albumin at the C-Termini

Variants of albumin were prepared using techniques known to the skilled person. A summary is provided below.

### (i) Plasmid Construction

Albumin variants were prepared using the amino acid sequence of HSA (SEQ ID NO: 2) as a ‘first parent albumin’ and the amino acid sequences of a number of different albumins each as a ‘second parent albumin’. In order to generate albumins according to the invention, the C-terminal string of amino acids from position 573 to 585 (KKLVAASQAALGL) (SEQ ID NO: 2) in HSA were mutated to those in macaque (PKFVAASQAALA) (SEQ ID NO: 6), mouse (PNLVTRCKDALA) (SEQ ID NO: 9), rabbit (PKLVESSKATLG) (SEQ ID NO: 14) and sheep (PKLVASTQAALA) (SEQ ID NO: 16) serum albumin. The codons used to introduce each amino acid substitution are given in Table 2. Table 3 summarises the plasmids constructed along with the amino acid substitutions made to HSA in order to generate albumins according to the invention.

TABLE 2
Codons used to introduce amino
acid substitutions into HSA

| Amino acid | Codon |
|---|---|
| Gly | GGT |
| Glu | GAA |
| Asp | GAT |
| Val | GTT |
| Ala | GCT |
| Arg | AGA |
| Lys | AAA |
| Asn | AAT |
| Met | ATG |
| Ile | ATT |
| Thr | ACT |
| Trp | TGG |
| Cys | TGT |
| Tyr | TAT |
| Leu | TTG |
| Phe | TTT |
| Ser | TCT |
| Gln | CAA |
| His | CAT |
| Pro | CCA |
| Stop | TAA |

TABLE 3
Variants of albumin at the C-terminus

| | Amino acid | | SEQ ID NO: of |
|---|---|---|---|
| Plasmid | substitution in albumin | Description of albumin variant | albumin variant |
| pDB4110 | HSA K573P | HSA with mutation K573P (HSA K573P) | 3 |
| pDB4114 | HSA K573P/L575F/ | N-terminus of HSA, C-terminus of | 20 |
| | G584A | macaque albumin (HSA-MacC) | |
| pDB4115 | HSA K573P/K574N/ | N-terminus of HSA, C-terminus of | 21 |
| | A577T/A578R/S579C/ | mouse albumin (HSA-MouseC) | |
| | Q580K/A581D/G584A | | |
| pDB4116 | HSA K573P/A577E/ | N-terminus of HSA, C-terminus of | 22 |
| | A578S/Q580K/A582T | rabbit albumin (HSA-RabC) | |
| pDB4117 | HSA K573P/A578S/ | N-terminus of HSA, C-terminus of | 23 |
| | S579T/G584A | sheep albumin (HSA-SheepC) | |
| pDB4540 | Mac P573K/F575L/ | N-terminus of Macaque albumin, C- | 32 |
| | A584G/X585L | terminus of HSA (Mac-HSAC) | |
| pDB4541 | Mac K574N/F575L/ | N-terminus of Macaque albumin, C- | 33 |
| | A577T/A578R/S579C/ | terminus of Mouse albumin (Mac-MouseC) | |
| | Q580K/A581D | | |
| pDB4542 | Mac F575L/A577E/ | N-terminus of Macaque albumin, C- | 34 |
| | A578S/Q580K/A582T/ | terminus of Rabbit albumin (Mac-RabC) | |
| | A584G | | |
| pDB4543 | Mac F575L/A578S/ | N-terminus of Macaque albumin, C- | 35 |
| | S579T | terminus of Sheep albumin (Mac-SheepC) | |

(HSA: human serum albumin; Mac: macaque serum albumin; In pDB4540 X585L represents an insertion because macaque serum albumin is 584 amino acids long but HSA is 585 amino acids long)

To generate the HSA C-terminal variants described in Table 3, synthetic DNA fragments (SacI/SphI) were generated (DNA2.0 Inc, USA) by gene assembly (the nucleotide sequence of the synthetic fragment encoding unchanged amino acids (i.e. wild type) was identical to that in pDB3927) and were sub-cloned into SacI/SphI-digested pDB3927 to produce plasmids pDB4114 to 4117 (Table 3). The construction of plasmids pDB4114 to pDB4117 is described in WO2011/051489. pDB3927 is described in WO2010/092135 (incorporated herein by reference).

The macaque albumin C-terminal variants can be made in a manner analogous to the HSA C-terminal variants described above. However, another suitable method uses PCR and was used to make the macaque albumin C terminal variants as described in Method 3, below.

Expression plasmids were generated in vivo (i.e. via homologous recombination in S. cerevisiae; a technique referred to as gap repair or in vivo cloning—see Orr-Weaver & Szostak. 1983. Proc. Natl. Acad. Sci. USA. 80:4417-4421). Modified plasmids listed in Table 3 were digested with BstEII/BsrBI or NsiI/PvuI and the linearised DNA molecules were purified using standard techniques. One hundred ng of each BstEII/BsrBI or NsiI/PvuI digested DNA, purified using a Qiagen PCR-Purification kit following the manufacturer's instructions, was mixed individually with 100 ng Acc65I/BamHI-digested pDB3936 (disclosed in WO 2010/092135) and used to directly transform S. cerevisiae BXP10cir⁰ as described below.

(ii) Transformation of S. cerevisiae

S. cerevisiae BXP10cir⁰ (as previously described WO2001/079480) was streaked on to YEPD plates (1% (w/v) yeast extract, 2% (w/v) Bactopeptone, 2% (w/v) glucose), 1.5% agar) and allowed to grow for 4 days at 30° C. prior to transformation. For gap repair, 100 ng BstEII/BsrBI-or NsiI/PvuI digested plasmids, containing the albumin variant expression cassette, and 100 ng Acc65I/BamHI digested pDB3936 were used to transform S. cerevisiae using a Sigma Yeast Transformation kit using a modified lithium acetate method (Sigma Yeast Transformation kit, YEAST-1, protocol 2; Ito et al. (1983) J. Bacteriol., 153, 16; Elble, (1992) Biotechniques, 13, 18). The protocol was amended slightly by incubating the transformation at room temperature for 4 h prior to heat shock. Following heat shock, the cells were briefly centrifuged before being re-suspended in 200 μl 1M sorbitol then spread over BMMD agar plates, the composition of BMMD is described by Sleep et al., (2001), Yeast, 18, 403. Plates were incubated at 30° C. for 4 days before individual colonies were patched on to fresh BMMD plates.

Stocks were prepared for each resultant yeast strain as follows: 10 ml BMMD broth in a 50 ml shake flask was inoculated with a heavy loop of each yeast patch and grown for 24 h at 30° C. with orbital shaking at 200 rpm. Cells were harvested by centrifugation at 1900×g for 5 min in a Sorval RT600 centrifuge, 5 mL supernatant was removed and replaced by trehalose 40% (w/v). The cells were resuspended and transferred to cyrovials (1 mL) for storage at −80° C.

(iii) Shake Flask Growth of S. cerevisiae

BMMD (recipe 0.17% (w/v) yeast nitrogen base without amino acid and ammonium sulphate (Difco), 37.8 mM ammonium sulphate, 29 mM citric acid, 142 mM disodium hydrogen orthophosphate dehydrate pH6.5, 2% (w/v) glucose) media (10 mL) was inoculated with each yeast strain and grown for 48 h (2 days) at 30° C. with orbital shaking at 200 rpm. An aliquot of each starter culture (4 mL) was used to inoculate 2×200 mL BMMD media in 500 ml shake flasks and grown for 96 h (4 days) at 30° C. with orbital shaking at 200 rpm. Cells were harvested by filtration through 0.2 μm vacuum filter membranes (Stericup, Millipore) including a GF-D prefilter (Whatman) and the supernatant retained for purification.

### (iv) Primary Concentration

Retained culture supernatant was concentrated using Tangential Flow Filtration using a Pall Filtron LV system fitted with a Omega 10 KD (0.093 m²) filter (LV Centramate™ cassette, Pall Filtron) with a transmembrane pressure of 20 psi and a recirculation rate of 180 mL.min⁻¹.

### (v) GP-HPLC Quantitation

Purified albumin variants were analysed by GP-HPLC and quantification as follows. Injections of 25 μL were made onto a 7.8 mm id×300 mm length TSK G3000SWXL column (Tosoh Bioscience), with a 6.0 mm id×40 mm length TSK SW guard column (Tosoh Bioscience). Samples were chromatographed in 25 mM sodium phosphate, 100 mM sodium sulphate, 0.05% (w/v) sodium azide, pH 7.0 at 1 mL/min, Samples were quantified by UV detection at 280 nm, by peak area, relative to a recombinant human albumin standard of known concentration (10 mg/mL) and corrected for their relative extinction coefficients. The same method can be used to analyse albumin fusions and conjugates.

(vi) Purification of Albumin Variants from Shake Flask

Albumin variants were purified from shake flask (either culture supernatant or concentrated culture supernatant) using a single chromatographic step using an albumin affinity matrix (AlbuPure®, ProMetic BioSciences, Inc.). Chromatography was performed at a constant linear velocity of 240 cm/h throughout. Culture supernatant was applied to a 6 cm bed height, 2.0 mL packed bed pre-equilibrated with 50 mM sodium acetate pH5.3. Following load the column was washed with 10 column volume (CV) of equilibration buffer, then 50 mM ammonium acetate pH8.0 (10 CV). Product was eluted with either 50 mM ammonium acetate 10 mM octanoate pH8.0, 50 mM ammonium acetate 30 mM sodium octanoate 200 mM sodium chloride pH7.0 or 200 mM potassium thiocyanate. The column was cleaned with 0.5M NaOH (3 cv) and 20 mM NaOH (3.5 cv). Eluate fractions from each albumin variant were concentrated and diafiltered against 10 volumes of 50 mM sodium chloride (Vivaspin20 10,000 MWCO PES with optional diafiltration cups, Sartorius). Purified albumin variants were quantified by GP-HPLC as described above.

Method 2: Determination of Receptor (shFcRn) Binding Properties of Albumin Variants

shFcRn was produced according to the methods of WO 2011/051489. Methods for the generation of shFcRn expression plasmids, expression and purification of shFcRn heterodimer can also be found in Berntzen et al. (2005) J. Immunol. Methods 298:93-104) and Andersen et al. (2010) J. Biol. Chem., 285:4826-4836.

SPR experiments were carried out using a Biacore 3000 instrument (GE Healthcare). Flow cells of CM5 sensor chips were coupled with shFcRn-GST (1500-2500 RU) using amine coupling chemistry as described in the protocol provided by the manufacturer. The coupling was performed by injecting 10 μg/ml of the protein in 10 mM sodium acetate pH 5.0 (GE Healthcare). Phosphate buffer (67 mM phosphate buffer, 0.15 M NaCl, 0.005% Tween 20) at pH 6.0) was used as running buffer and dilution buffer. Regeneration of the surfaces were done using injections of HBS-EP buffer (0.01 M HEPES, 0.15 M NaCl, 3 mM EDTA, 0.005% surfactant P20) at pH 7.4 (Biacore AB). For binding to immobilized shFcRn-GST, 1.0-0.5 mM of each HSA derivative or variant was injected over the surface at constant flow rate (40 μl/ml) at 25° C. In all experiments, data was zero adjusted and the reference cell subtracted. Data evaluation was performed using BIAevaluation 4.1 software (BIAcore AB).

### Method 3: Preparation of Macaque Albumin C-Terminal Variants

Expression cassettes for macaque albumin C-terminal variants (macaque albumin (1 to 572) and the C-terminal amino acids from human (573 to 585 of SEQ ID No: 2; KKLVAASQAALGL), mouse (573 to 584 (which correspond to 597 to 608 of the immature sequence i.e. SEQ ID No: 9); PNLVTRCKDALA), rabbit (573 to 584 (which correspond to 597 to 608 of the immature sequence i.e. SEQ ID No: 14); PKLVESSKATLG) or sheep (573 to 583 (which correspond to 596 to 607 of the immature sequence i.e. SEQ ID No 16); PKLVASTQAALA) albumins) were prepared by replacing the C-terminal region of macaque albumin [the ‘first albumin’, SEQ ID No: 6] with that of the C-terminal region of the ‘second albumin’ (e.g. human, mouse, rabbit and sheep) (Table 3).

Specifically, the nucleotide sequence encoding the C-terminal amino acids from the albumins described above were amplified by PCR using oligonucleotide pairs and template DNAs described in Tables 4 and 5. Phusion polymerase (New England Biolabs) was used for all PCRs following the manufacturer's instructions. PCR-fragments were digested with SpeI/HindIII and ligated into SpeI/HindIII-digested pDB4118 using standard techniques.

TABLE 4
Oligonucleotides and template DNAs
used to generate PCR fragments

| | Oligo | Oligo | | Size |
|---|---|---|---|---|
| Construct | (Fwd) | (Rev) | Template | (bp) |
| N-terminus of macaque albumin, | xAP321 | xAP317 | pDB3927 | 190 |
| C-terminus of HSA | | | | |
| N-terminus of macaque albumin, | xAP321 | xAP318 | pDB4115 | 189 |
| C-terminus of mouse albumin | | | | |
| N-terminus of macaque albumin, | xAP321 | xAP319 | pDB4116 | 189 |
| C-terminus of rabbit albumin | | | | |
| N-terminus of macaque albumin, | xAP321 | xAP320 | pDB4117 | 189 |
| C-terminus of sheep albumin | | | | |

Plasmid pDB3927 is described in WO2010/092135 (incorporated herein by reference). Plasmids pDB4115 to pDB4117 are disclosed in WO2011/051489 (incorporated herein by reference).

TABLE 5
Oligonucleotide sequences

| Oligonu- | | SEQ |
|---|---|---|
| cleotide | Sequence (5′ - 3′) | ID No. |
| xAP317 | ATTAATTAAGCTTATTATAAGCCTAAGGCAGCTTGACTTGCAGCAA | 38 |
| | CAAGTTTTTTCCCTTCCTCTGCAAAACATGCTTCCTTGTCATCAGC | |
| xAP318 | ATATTAATTAAGCTTATTAAGCCAAAGCATCTTTACATCTAGTAACC | 39 |
| | AAATTTGGCCCTTCCTCTGCAAAACATGCTTCCTTGTCATCAGC | |
| xAP319 | ATATTAATTAAGCTTATTAACCCAAAGTAGCTTTAGAAGATTCAACC | 40 |
| | AATTTAGGCCCTTCCTCTGCAAAACATGCTTCCTTGTCATCAGC | |
| xAP320 | ATATTAATTAAGCTTATTAAGCCAAAGCAGCTTGAGTAGAAGCAAC | 41 |
| | CAATTTAGGCCCTTCCTCTGCAAAACATGCTTCCTTGTCATCAGC | |
| xAP321 | CTTTAGTAGAACTAGTAAAACACAAGC | 42 |

A plasmid for the expression of macaque albumin was prepared as follows. A 1.780 kb Bg/II/HindIII synthetic DNA fragment (containing 3′ region of the DNA-encoding the modified fusion leader (disclosed in WO 2010/092135), DNA sequence encoding macaque albumin (codon optimised for expression in S. cerevisiae) and the 5′ region of the mADHt) was generated by gene assembly (DNA2.0, USA, SEQ ID NO: 36). The synthetic Bg/II/HindIII fragment was cloned into Bg/II/HindIII digested pDB4081 to produce pDB4118.

pDB4081 was made by the ligation of a synthetic DNA fragment, BsaI/SphI digested, which had been generated by gene assembly (DNA2.0 Inc, USA) (SEQ ID NO: 37, containing 3′ region of the PRB1 promoter, modified fusion leader sequence, nucleotide sequence encoding HSA and 5′ region of the modified ADH1 terminator) into HindIII/SphI-digested pDB4005 (disclosed in WO 2011/051489).

The final macaque expression plasmid was generated by in vivo cloning/gap-repair as follows—plasmids pDB4118 was digested with BstEII/BsrBI, purified using a Qiagen PCR-Purification kit following the manufacturer's instructions, and 100 ng of the purified DNA was combined with 100 ng Acc65I/BamHI-digested pDB3936 and used to co-transform S. cerevisiae BXP10cir⁰ as described previously.

Ligations were used to transform E. coli DH5α using standard techniques and resulting plasmids pDB4540, pDB4541, pDB4542 and pDB4543 are listed in Table 2. Plasmids pDB4540 to 44543 were digested with NsiII/PvuI and purified using a Qiagen PCR Purification kit following the manufacturer's instructions. Purified NsiII/PvuI-digested plasmids (100 ng), along with 100 ng Acc65I/BamHI-digested pDB3936, were used to directly co-transform S. cerevisiae BXP10cir⁰ by gap-repair as described in above. Trehalose stocks were prepared for yeast stains as described above.

### Method 4: Construction of Truncated HSA Mutants

Expression constructs for truncated HSA mutants (Table 6, below) were generated by PCR and gap-repair. This was achieved by generating PCR products using Phusion Polymerase (New England Biolabs), according to the manufacturer's instruction, pDB3927 (described in WO2010/092135 (incorporated herein by reference) and oligonucleotides (Table 6 and 7). This resulted in DNAs in which specific codons (i.e. amino acids 568 and 572 to 585, excluding position 573) were replaced with the translational stop codon amino TAA. These PCR products were cloned into plasmids and used to form expression plasmids in yeast by gap repair.

TABLE 6
Truncated HSA molecules

| Molecule | Oligonucleotide pair | Plasmid |
|---|---|---|
| HSA 585stop | xAP265/xAP294 | pDB4544 |
| HSA 584stop | xAP265/xAP295 | pDB4545 |
| HSA 583stop | xAP265/xAP296 | pDB4546 |
| HSA 582stop | xAP265/xAP297 | pDB4547 |
| HSA 581stop | xAP314/xAP298 | pDB4548 |
| HSA 580stop | xAP314/xAP299 | pDB4549 |
| HSA 579stop | xAP314/xAP300 | pDB4550 |
| HSA 578stop | xAP314/xAP301 | pDB4551 |
| HSA 577stop | xAP314/xAP302 | pDB4552 |
| HSA 576stop | xAP314/xAP303 | pDB4553 |
| HSA 575stop | xAP314/xAP304 | pDB4554 |
| HSA 574stop | xAP314/xAP305 | pDB4555 |
| HSA 572stop | xAP314/xAP306 | pDB4556 |
| HSA 568stop | xAP314/xAP307 | pDB4557 |

In Table 6, albumin variants are named such that ‘HSA 585stop’ is an HSA variant in which the native amino acid at position 585 is substituted with a stop codon.

Specifically, for HSA568stop expression construct oligonucleotides xAP314 and xAP307 were used to amplify a 493 bp fragment from pDB3927, containing DNA sequence encoding HSA DIII, according to the manufacturer's instructions. A stop codon was engineered into oligonucleotide xAP307 so that translation of the DNA sequence encoding HSA terminated following amino acid 567. The PCR-fragment was digested with AvrII/Bsu36I purified using a Qiagen PCR-clean up kit (according to the manufacturer's instructions) and ligated into AvrII/Bsu361-digested pDB3927. Ligations were transformed into E. coli DH5α, subsequent plasmids isolated from transformants using a Qiagen miniprep kit (according to the manufacturer's instructions) and the correct constructs identified by restriction analysis. This produced the HSA568stop expression construct pDB4557.

The HSA572stop and HSA574stop to HSA581stop expression constructs were made in the same manner as the HSA568stop construct using the oligonucleotides as listed in Table 7 to produce plasmids pDB4548 to pDB4556 (Table 6).

For the HSA582 to HSA585stop constructs (1.122 kb) fragments were PCR amplified from pDB3927 using oligonucleotides listed in Table 7. The PCR-fragments were each digested with Bg/II/HindIII isolated and ligated into pDB2923 (Finnis, C. J. et al. (2010). High-level production of animal-free recombinant transferrin from Saccharomyces cerevisiae. Microb Cell Fact 9, 87) to produce plasmids #10D, #11B, #12C and #13D, respectively. Plasmids #10D to #13D were digested with AvrII/SphI and a 666 bp fragments (containing the DNA encoding the C-terminal end of albumin) were isolated from each and ligated into AvrII/SphI-digested pDB3927 to produce the gap-repair constructs pDB4544-pDB4547, respectively (Table 6).

Plasmids pDB4544-pDB4557 were digested with NsiI/PvuI, the DNA was purified using a Qiagen PCR Purification kit as per the manufacturer's instructions, before being used, along with Acc651/BamHI-digested pDB3936, to co-transform S. cerevisiae BXP10cir⁰ as described above generating expression plasmids in the yeast by gap-repair.

Stocks were prepared for each resultant yeast strain as described previously.

The strain producing the HSA573stop variant had been made previously, as described in the WO2011/0541489 (incorporated herein by reference).

TABLE 7
Oligonucleotide sequences for preparation of truncated
HSA mutants

| Oligonu- | | SEQ |
|---|---|---|
| cleotide | Sequence (5′ - 3′) | ID No: |
| xAP265 | GCTCGCCTGAGCCAGAG | 43 |
| xAP294 | GAATTAAGCTTATTATTAGCCTAAGGCAGC | 44 |
| xAP295 | GAATTAAGCTTATTATAATTATAAGGCAGC | 45 |
| xAP296 | GAATTAAGCTTATTATAAGCCTTAGGCAGCTTG | 46 |
| xAP297 | GAATTAAGCTTATTATAAGCCTAATTAGGCTTGACTTGC | 47 |
| xAP298 | GAATTAAGCTTATTATAAGCCTAAGGCTTATTGACTTGCAGCAACA | 48 |
| | AG | |
| xAP299 | GAATTAAGCTTATTATAAGCCTAAGGCAGCTTAACTTGCAGCAAC | 49 |
| | AAG | |
| xAP300 | GAATTAAGCTTATTATAAGCCTAAGGCAGCTTGTTATGCAGCAACA | 50 |
| | AG | |
| xAP301 | GAATTAAGCTTATTATAAGCCTAAGGCAGCTTGACTTTAAGCAACA | 51 |
| | AG | |
| xAP302 | GAATTAAGCTTATTATAAGCCTAAGGCAGCTTGACTTGCTTAAACA | 52 |
| | AGTTTTTTAC | |
| xAP303 | GAATTAAGCTTATTATAAGCCTAAGGCAGCTTGACTTGCAGCTTAA | 53 |
| | AGTTTTTTAC | |
| xAP304 | GAATTAAGCTTATTATAAGCCTAAGGCAGCTTGACTTGCAGCAAC | 54 |
| | TTATTTTTTACCCTC | |
| xAP305 | GAATTAAGCTTATTATAAGCCTAAGGCAGCTTGACTTGCAGCAAC | 55 |
| | AAGTTATTTACCCTC | |
| xAP306 | GAATTAAGCTTATTATAAGCCTAAGGCAGCTTGACTTGCAGCAAC | 56 |
| | AAGTTTTTTTTACTCCTC | |
| xAP307 | GAATTAAGCTTATTATAAGCCTAAGGCAGCTTGACTTGCAGCAAC | 57 |
| | AAGTTTTTTACCCTCCTCGGCTTAGCAGG | |
| xAP314 | CTCAAGAAACCTAGGAAAAGTGGGCAGC | 58 |

### Example 1

### Variants of Albumin Comprising the N-Terminal of HSA and the C-Terminus of HSA, Macaque Albumin, Mouse Albumin, Rabbit Albumin or Sheep Albumin

The following variants were generated and binding to the shFcRn was determined as described in Methods 1 and 2. The results are presented in Table 8

TABLE 8
Kinetics of the HSA C-terminal swapped variant interactions with shFcRn.

| | | N-region | | | | | | |
|---|---|---|---|---|---|---|---|---|
| | | (“recipient”/first | | C-region (“donor”/ | | | | |
| | | albumin) | | second albumin) | | | | |
| | | | Residue | | Residue | | | |
| | SEQ | | numbers | | numbers | | | |
| Albumin | ID | Source | of | Source | of | ka | | KD^{b} |
| variant^{a} | NO: | species | variant | species | variant | (10³/Ms) | kd (10⁻³/s) | (μM) |
| wt HSA | 2 | n/a | n/a | n/a | n/a | 4.4 ± 0.0 | 24.0 ± 0.1 | 5.4 |
| HSA K573P | 3 | n/a | n/a | n/a | n/a | 2.8 ± 0.0 | 0.4 ± 0.0 | 0.1 |
| MacSA | 6 | n/a | n/a | n/a | n/a | 3.1 ± 0.1 | 8.6 ± 0.1 | 2.7 |
| HSA-MacC | 20 | Human | 1-572 | Macaque | 573-584 | 4.1 ± 0.1 | 5.6 ± 0.0 | 1.3 |
| MouseSA | 9 | n/a | n/a | n/a | n/a | 3.8 ± 0.0 | 3.1 ± 0.1 | 0.8 |
| HSA-MouseC | 21 | Human | 1-572 | Mouse | 573-584 | 3.7 ± 0.1 | 1.3 ± 0.0 | 0.3 |
| RabbitSA | 14 | n/a | n/a | n/a | n/a | 1.9 ± 0.3 | 1.7 ± 0.1 | 0.9 |
| HSA-RabC | 22 | Human | 1-572 | Rabbit | 573-584 | 3.5 ± 0.0 | 1.6 ± 0.0 | 0.4 |
| SheepSA | 16 | n/a | n/a | n/a | n/a | ND | ND | ND |
| HSA-SheepC (*) | 23 | Human | 1-572 | Sheep | 572-583 | 3.3 ± 0.0 | 2.1 ± 0.0 | 0.6 |

^{a}Dilutions of HSA variants were injected over immobilized shFcRn (~1500 RU).
^{b}The kinetic rate constants were obtained using a simple first-order (1:1) bimolecular interaction model.
Not determined due to weak binding (ND)
(*) Total length of HSA-SheepC is 584 amino acids.

This example demonstrates that for all C-terminal swaps to human albumin tested an increase in binding over the donor albumin and an increase in binding over the recipient albumin (i.e. HSA) was observed. All donor sequences contain Pro at position 573. This shows that swapping the C-terminus of albumin allows modulation of the binding affinity of the polypeptide, thus allowing the binding affinity to be ‘tailored’ according to requirement.

### Example 2 (Comparative Example)

### Truncation of the C-Terminal End of HSA Modulates Binding to shFcRn

The following albumin variants were prepared and binding to the shFcRn determined as described in Methods 4 and 2, respectively, above. The results are presented in Table 9 and FIG. 4.

TABLE 9
Truncation of the C-terminal end of HSA
modulates binding to shFcRn at pH 6.0

| Albumin variant^{a} | Ka (10³/Ms) | kd (10⁻³/s) | KD^{b} (μM) | KD^{c} (μM) |
|---|---|---|---|---|
| WT | 6.6 ± 0.1 | 9.1 ± 0.1 | 1.3 | 2.4 |
| 584Stop | 8.6 ± 0.0 | 32.0 ± 0.1 | 3.7 | ND |
| 582Stop | 13.0 ± 0.2 | 65.0 ± 0.0 | 5.0 | ND |
| 581Stop | 3.6 ± 0.0 | 32.0 ± 0.1 | 9.0 | ND |
| 580Stop | 9.8 ± 0.1 | 60 ± 0.0 | 6.1 | 13.2 |
| 579Stop | ND | ND | ND | 17.0 |
| 578Stop | ND | ND | ND | 19.9 |
| 577Stop | ND | ND | ND | 23.0 |
| 573Stop | ND | ND | ND | 14.1 |
| 572Stop | ND | ND | ND | 10.4 |
| 568Stop | ND | ND | ND | 23.0 |

^{a}Dilutions of HSA variants were injected over immobilized shFcRn (~2000 RU).
^{b}The kinetic rate constants were obtained using a simple first-order (1:1) bimolecular interaction model. The kinetic values represent the average of duplicates.
^{c}The steady state affinity constant was obtained using an equilibrium (Req) binding model supplied by the BIAevaluation 4.1 software.
d: Not determined (ND).

The data of Table 9 and FIG. 4 show the importance of the C-terminus of HSA in pH dependent binding to shFcRn. Surprisingly, removal of the last amino acid of HSA (Leu585) reduced binding to the receptor by 50% compared with wt HSA and further truncation increased the effect (FIG. 4). Determination of KDs shows the dramatic impact of C-terminal truncations (Table 9).

FIG. 5 shows a general trend in which increasing the extent of C-terminal truncation of an albumin variant decreases the binding affinity of the albumin variant to FcRn relative to the binding affinity of wild-type HSA to FcRn.

These data show that removal of from 1 to 17 amino acids from the C-terminal of albumin reduces the binding affinity of albumin to FcRn. This shows that the C-terminus of albumin has an important role in binding of albumin to FcRn.

HSA is 585 amino acids long. In contrast animal albumins are typically shorter e.g. macaque, mouse and rabbit HSA are each 584 amino acids long and sheep albumin is 583 amino acids long. Example 1 shows that the binding affinity of each of macaque, mouse and rabbit albumin to human FcRn is stronger than the binding affinity of HSA to human FcRn. Also, the binding of chimeras of HSA and macaque, mouse, rabbit or sheep albumin to human FcRn is stronger than the binding of wild-type human, macaque, mouse, rabbit or sheep albumin to human FcRn again indicating the importance of the extreme C terminus of albumin. The data of Example 2 show that removal of the C terminus of HSA reduces binding affinity to FcRn. The data of Example 1 show that the binding affinity of truncated albumin can be restored by replacing the C-terminus of HSA with the C-terminus of another albumin, such as an animal albumin as described herein. All of the tested animal albumins have Pro at position 573. In contrast HSA has Lys at position 573. This suggests that 573 has an important positive role in the binding of albumin to FcRn.

The data of Examples 1 and 2 indicate that removal of the C-terminus of a first albumin (e.g. HSA) and replacing it with the C-terminus of a second albumin (e.g. an animal albumin) results in a chimeric albumin having altered FcRn-binding affinity compared with the FcRn binding affinity of the first albumin demonstrating the importance of the C-terminus of albumin in binding to human FcRn. The chimera-FcRn binding affinity is stronger than the binding affinity of the C-terminal truncated albumin to FcRn. However, since the binding affinity of the chimera is different to both that of the first and the second albumin, it seems that parts of albumin other than the C-terminus are also involved in binding to human FcRn.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A polypeptide which comprises:
(i) an N-terminal region of a first albumin, albumin variant or fragment thereof; and
(ii) a C-terminal region of a second albumin, albumin variant or fragment thereof in which:
(a) the N-terminal of the first albumin, albumin variant or fragment thereof comprises the amino acids of the molecule from which it is derived except the C-terminal 1 to 100 amino acids; and
(b) the C-terminal of the second albumin, albumin variant or fragment thereof comprises the C-terminal 1 to 100 amino acids of the second albumin, albumin variant or fragment thereof; and
(c) the polypeptide has (i) an altered half-life compared with the first albumin, albumin variant or fragment thereof and/or (ii) an altered binding affinity to FcRn compared with the first albumin, albumin variant or fragment thereof.

2. The polypeptide according to claim 1 which comprises:
(i) an N-terminal region of a first albumin, albumin variant or fragment thereof; and
(ii) a C-terminal region of a second albumin, albumin variant or fragment thereof in which:
(a) the N-terminal of the first albumin, albumin variant or fragment thereof comprises 83 to 100% of the albumin, albumin variant or fragment from which it is derived; and
(b) the C-terminal of the second albumin, albumin variant or fragment thereof comprises the C-terminal 0.5% to 17% amino acids of the albumin, albumin variant or fragment
(c) the polypeptide has (i) an altered half-life compared with the first albumin, albumin variant or fragment thereof and/or (ii) an altered binding affinity to FcRn compared with the first albumin, albumin variant or fragment thereof.

3. The polypeptide according to claim 1 wherein the first albumin, to which the half-life and/or FcRn-binding affinity of the polypeptide is compared, is a wild-type albumin or a naturally occurring albumin, preferably HSA (SEQ ID No. 2).

4. The polypeptide according to claim 1 in which the N-terminal of the first albumin, albumin variant or fragment thereof comprises all amino acids of the molecule from which it is derived except the C-terminal 2 to 30 amino acids.

5. The polypeptide according to claim 1 in which the N-terminal of the first albumin, albumin variant or fragment thereof comprises all amino acids of the molecule from which it is derived except the C-terminal 12 to 20 amino acids, most preferably the C-terminal 13 amino acids.

6. The polypeptide according to claim 1 in which the N-terminal of the first albumin, albumin variant or fragment thereof comprises at least 97% of the albumin, albumin variant or fragment from which it is derived.

7. The polypeptide according to claim 1 in which the N-terminal of the first albumin, albumin variant or fragment thereof comprises at least 98% of the albumin, albumin variant or fragment from which it is derived.

8. The polypeptide according to claim 1 in which the C-terminal of the second albumin, albumin variant or fragment thereof comprises the C-terminal 2 to 30 amino acids, more preferably the C-terminal 12 to 20 amino acids, most preferably the C-terminal 13 amino acids.

9. The polypeptide according to claim 1 in which the C-terminal of the second albumin, albumin variant or fragment thereof comprises the C-terminal 1 to 3% of the second albumin, albumin variant or fragment thereof.

10. The polypeptide according to claim 1 in which the first albumin is selected from human albumin, macaque albumin, rabbit albumin, mouse albumin, sheep albumin, goat albumin, chimpanzee albumin, hamster albumin, guinea pig albumin, rat albumin, cow albumin, horse albumin, donkey albumin, dog albumin, chicken albumin, or pig albumin.

11. The polypeptide according to claim 1 in which the first albumin comprises or consists of domain III of an albumin.

12. The polypeptide according to claim 1 in which the second albumin is selected from macaque albumin, mouse albumin, rabbit albumin, sheep albumin, human albumin, goat albumin, chimpanzee albumin, hamster albumin, guinea pig albumin, rat albumin, cow albumin, horse albumin, donkey albumin, dog albumin, chicken albumin, or pig albumin.

13. The polypeptide according to claim 1 which has a longer half-life and/or stronger binding affinity to FcRn compared with the first albumin, albumin variant or fragment thereof.

14. The polypeptide according to claim 1 which has a shorter half-life and/or weaker binding affinity to FcRn compared with the first albumin, albumin variant or fragment thereof.

15. The polypeptide according to claim 1 in which the first albumin or albumin variant has, compared to SEQ ID NO: 2, more than 80%, preferably more than 90%, more preferred more than 95%, more preferred more than 96%, even more preferred more than 97%, more preferred more than 98% and most preferred more than 99% identity over the length of the N-terminal region of the first albumin which is present in the polypeptide.

16. The polypeptide according to claim 1 in which the N-terminal region of the first albumin, albumin variant or fragment thereof comprises amino acids 1 to 565, 566, 567, 568, 569, 570, 571, 572, 573, 574 or 575 of SEQ ID NO: 2.

17. The polypeptide according to claim 1 in which the first albumin fragment:
(i) has, compared to SEQ ID NO: 24, SEQ ID NO: 25, SEQ ID NO: 26, SEQ ID NO: 27, SEQ ID NO: 28 or SEQ ID NO: 29, more than 80%, preferably more than 90%, more preferred more than 95%, more preferred more than 96%, even more preferred more than 97%, more preferred more than 98% and most preferred more than 99% identity over the N-terminal region of the first albumin which is present in the polypeptide; and/or
(ii) comprises at least 20, preferably at least 50, preferably at least 100, more preferred at least 200, more preferred at least 300, more preferred at least 400 and most preferred at least 500 sequential amino acids from a natural albumin such as SEQ ID NO: 2.

18. A polypeptide according to claim 1 in which the polypeptide comprises or consists of SEQ ID NO: 20, SEQ ID NO: 21, SEQ ID NO: 22 or SEQ ID NO: 23.

19. A fusion polypeptide comprising a polypeptide according to claim 1 and a fusion partner polypeptide.

20. A conjugate comprising the polypeptide according to claim 1 and a conjugation partner such as a pharmaceutically beneficial moiety such as a therapeutic moiety, a prophylactic moiety or a diagnostic moiety.

21. A composition comprising a polypeptide, fusion polypeptide or conjugate according to claim 1 and a pharmaceutically acceptable carrier.

22. The composition according to claim 21 comprising a compound comprising an antibody binding domain (ABD) and a pharmaceutically beneficial moiety such as a therapeutic moiety, a prophylatic moiety or a diagnostic moiety.

23. A polynucleotide encoding a polypeptide or fusion polypeptide according to claim 1.

24. A vector comprising a polynucleotide according to claim 23.

25. A host cell comprising a polynucleotide according to claim 23 or a vector according to claim 24.

26. A method of prophylaxis, treatment or diagnosis comprising administering a polypeptide, fusion polypeptide, conjugate, composition or polynucleotide according to claim 1 to a subject.

27. A method for preparing a variant of albumin, a fragment thereof or a fusion polypeptide comprising the variant or fragment, the method comprising:
i) providing a polynucleotide encoding an N-terminal region of a first albumin, albumin variant or fragment thereof and a C-terminal region of a second albumin, albumin variant or fragment thereof and optionally encoding a fusion partner polypeptide;
in which (a) the N-terminal of the first albumin, albumin variant or fragment thereof comprises amino acids of the molecule from which it is derived except the C-terminal 1 to 100 amino acids; and (b) the C-terminal of the second albumin, albumin variant or fragment thereof comprises the C-terminal 1 to 100 amino acids of the second albumin, albumin variant or fragment thereof; and (c) the polypeptide or fusion polypeptide encoded by the polynucleotide has an altered half-life compared with a polypeptide or fusion polypeptide comprising the first albumin, albumin variant or fragment and/or an altered binding affinity to FcRn compared with the first albumin, albumin variant or fragment thereof;
ii) expressing the polynucleotide in a host cell; and
iii) recovering the resultant polypeptide or fusion polypeptide.

28. A method for preparing a variant of albumin, a fragment thereof or a fusion polypeptide comprising the variant or fragment, according to claim 27, the method comprising:
i) providing a polynucleotide encoding an N-terminal region of a first albumin, albumin variant or fragment thereof and a C-terminal region of a second albumin, albumin variant or fragment thereof and optionally encoding a fusion partner polypeptide;
in which (a) the N-terminal of the first albumin, albumin variant or fragment thereof comprises 83 to 99.5% of the albumin, albumin variant or fragment from which it is derived; (b) the C-terminal of the second albumin, albumin variant or fragment thereof comprises the C-terminal a comprises the C-terminal 0.5% to 17% amino acids of the albumin, albumin variant or fragment of the second albumin, albumin variant or fragment thereof; and (c) the polypeptide or fusion polypeptide encoded by the polynucleotide has an altered half-life compared with a polypeptide or fusion polypeptide comprising the first albumin, albumin variant or fragment and/or an altered binding affinity to FcRn compared with the first albumin, albumin variant or fragment thereof;
ii) expressing the polynucleotide in a host cell; and
iii) recovering the resultant polypeptide or fusion polypeptide.

29. A method for altering the half-life of a molecule comprising:
(a) where the molecule is a polypeptide, fusing the molecule to a polypeptide according to claim 1 or conjugating the molecule to a polypeptide or fusion polypeptide according to claim 1 or associating the molecule with a polypeptide according to claim 1 or incorporating the molecule into a nanoparticle or microparticle comprising or consisting of a polypeptide according to claim 1;
(b) where the molecule is not a polypeptide, conjugating the molecule to a polypeptide or fusion polypeptide according to claim 1 or associating the molecule with a polypeptide according to claim 1 or incorporating the molecule into a nanoparticle or microparticle comprising or consisting of a polypeptide according to claim 1.

30. A nanoparticle or microparticle comprising a polypeptide according to claim 1, a fusion polypeptide according to claim 19 and/or a conjugate according to claim 20.

31. An associate comprising a polypeptide according to claim 1, a fusion polypeptide according to claim 19 and/or a conjugate according to claim 20 and a non-albumin moiety.

32. The fusion, conjugate, associate, composition, nanoparticle and/or microparticle of an albumin variant or derivative according to claim 1 wherein the fusion or conjugation or associate or composition, nanoparticle or microparticle comprises one or more moiety selected from those described herein.

33. (canceled)


SEQUENCE LISTING (58 sequences)

SEQ ID NO: 1 - DNA, 1758 bp, Homo sapiens
  misc_feature (1)..(1758): cDNA encoding HSA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 2 - protein, 585 aa, Homo sapiens [sequence omitted]

SEQ ID NO: 3 - protein, 585 aa, Artificial Sequence
  HSA K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 4 - protein, 609 aa, Homo sapiens [sequence omitted]

SEQ ID NO: 5 - protein, 621 aa, Pan troglodytes
MNESSCCSTSLPAFGVSVLDSGHSSSSAYSRGVFRRDAHKSEVAHRFKDLGEENFKALVLVAFAQYLQQCPFEDHVKLVNEVTEFAKTCVADESAENCDKSLHTLFGDKLCTVATLREKYGEMADCCAKQEPERNECFLQHKDDNPNLPRLVRPEVDVMCTAFHDNEGTFLKKYLYEVARRHPYFYAPELLFFAERYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKEVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 608 aa, Macaca mulatta
MKWVTFISLLFLFSSAYSRGVFRRDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 7 - protein, 608 aa, Mesocricetus auratus
MKWVTFLLLLFVSDSAFSRGLFRRDAHKSEIAHRFKDLGEQHFKGLVLIAFSQFLQKCPYEEHVKLVNEVTDFAKTCVADESAENCDKSLHTLFGDKLCAIPTLRDSYGELADCCAKKEPERNECFLKHKDDHPNLPPFVRPDAEAMCTSFQENAVTFMGHYLHEVARRHPYFYAPELLYYAEKYSAIMTECCGEADKAACITPKLDALKEKALASSVNQRLKCSSLQRFGQRAFKAWAVARMSQKFPKADFAEITKLATDLTKLTEECCHGDLLECADDRAELAKYMCENQASISSKLQACCDKPVLKKSHCLSEVENDDLPADLPSLAADFVEDKEVCKNYAEAKDVFLGTFLYEYARRHPDYSVALLLRLAKKYEATLEKCCAEADPSACYGKVLDEFQPLVEEPKNLVKANCELFEKLGEYGFQNALIVRYTQKAPQVSTPTLVEAARNLGKVGSKCCVLPEAQRLPCVEDYISAILNRVCVLHEKTPVSEQVTKCCTGSVVERRPCFSALPVDETYVPKEFKAETFTFHADICSLPEKEKQMKKQAALVELVKHKPKATGPQLRTVLGEFTAFLDKCCKAEDKEACFSEDGPKLVASSQAALA

SEQ ID NO: 8 - protein, 608 aa, Cavia porcellus
MKWVTFISLLFLFSSVYSRGVFRREAHKSEIAHRFNDLGEGHFKGLVLITLSQHLQKSPFEEHVKLVNEVTDFAKACVADESAQNCGKAIATLFGDKVCAIPSLRETYGELADCCAKEDPDRVECFLQHKDDNPNLPPFERPEPEALCTAFKENNDRFIGHYLYEVSRRHPYFYAPELLYYAEKYKNALTECCEAADKAACLTPKLDAIKEKALVSSAQQRLKCASLQKFGERAFKAWSVARLSQKFPKAEFAEISTIVTSLTKVTKECCHGDLLECADDRQELAKYMCEHQDSISSKLKECCVKPTLQKAHCILEIQRDELPTELPDLAVDFVEDKEVCKNFAEAKDVFLGTFLYEYSRRHPEYSIGMLLRIAKGYEAKLEKCCAEADPHACYAKVFDELQPLIDEPKKLVQQNCELFDKLGEYGFQNALAVRYTQKAPQVSTPTLVEYARKLGSVGTKCCSLPETERLSCTENYLALILNRLCILHEKTPVSERVTKCCTESLVNRRPCFSALHVDETYVPKPFHADSFTFHADICTLPEKEKQVKKQMALVELVKHKPKASEEQMKTVMGDFAAFLKKCCDADNKEACFTEDGPKLVAKCQATLA

SEQ ID NO: 9 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 10 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 11 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 12 - protein, 607 aa, Equus caballus
MKWVTFVSLLFLFSSAYSRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKLACLIPKLDALKERILLSSAKERLKCSSFQNFGERAVKAWSVARLSQKFPKADFAEVSKIVTDLTKVHKECCHGDLLECADDRADLAKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYRTVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYVPKEFKAETFTFHADICTLPEDEKQIKKQSALAELVKHKPKATKEQLKTVLGNFSAFVAKCCGREDKEACFAEEGPKLVASSQLALA

SEQ ID NO: 13 - protein, 607 aa, Equus asinus
MKWVTFVSLLFLFSSAYFRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKAGCLIPKLDALKERILLSSAKERLKCSSFQKFGERAFKAWSVARLSQKFPKADFAEVSKIVTDLTKVHKECCHGDLLECADDRADLTKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYATVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYIPKEFKAETFTFHADICTLPEDEKQIKKQSALAELVKHKPKATKEQLKTVLGNFSAFVAKCCGAEDKEACFAEEGPKLVASSQLALA

SEQ ID NO: 14 - protein, 608 aa, Oryctolagus cuniculus
MKWVTFISLLFLFSSAYSRGVFRREAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 15 - protein, 583 aa, Capra hircus
DTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLKHKDDSPDLPKLKPEPDTLCAEFKADEKKFWGKYLYEVARRHPYFYAPELLYYANKYNGVFQECCQAEDKGACLLPKIETMREKVLASSARQRLRCASIQKFGERALKAWSVARLSQKFPKADFTDVTKIVTDLTKVHKECCHGDLLECADDRADLAKYICDHQDTLSSKLKECCDKPVLEKSHCIAEIDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDGESFTFHADICTLPDTEKQIKKQTALVELLKHKPKATDEQLKTVMENFVAFVDKCCAADDKEGCFLLEGPKLVASTQAALA

SEQ ID NO: 16 - protein, 607 aa, Ovis aries
MKWVTFISLLLLFSSAYSRGVFRRDTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLNHKDDSPDLPKLKPEPDTLCAEFKADEKKFWGKYLYEVARRHPYFYAPELLYYANKYNGVFQECCQAEDKGACLLPKIDAMREKVLASSARQRLRCASIQKFGERALKAWSVARLSQKFPKADFTDVTKIVTDLTKVHKECCHGDLLECADDRADLAKYICDHQDALSSKLKECCDKPVLEKSHCIAEVDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDEKFFTFHADICTLPDTEKQIKKQTALVELLKHKPKATDEQLKTVMENFVAFVDKCCAADDKEGCFVLEGPKLVASTQAALA

SEQ ID NO: 17 - protein, 608 aa, canis lupus familiaris
MKWVTFISLFFLFSSAYSRGLVRREAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYARRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCAEDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 18 - protein, 615 aa, Gallus gallus
MKWVTLISFIFLFSSATSRNLQRFARDAEHKSEIAHRYNDLKEETFKAVAMITFAQYLQRCSYEGLSKLVKDVVDLAQKCVANEDAPECSKPLPSIILDEICQVEKLRDSYGAMADCCSKADPERNECFLSFKVSQPDFVQPYQRPASDVICQEYQDNRVSFLGHFIYSVARRHPFLYAPAILSFAVDFEHALQSCCKESDVGACLDTKEIVMREKAKGVSVKQQYFCGILKQFGDRVFQARQLIYLSQKYPKAPFSEVSKFVHDSIGVHKECCEGDMVECMDDMARMMSNLCSQQDVFSGKIKDCCEKPIVERSQCIMEAEFDEKPADLPSLVEKYIEDKEVCKSFEAGHDAFMAEFVYEYSRRHPEFSIQLIMRIAKGYESLLEKCCKTDNPAECYANAQEQLNQHIKETQDVVKTNCDLLHDHGEADFLKSILIRYTKKMPQVPTDLLLETGKKMTTIGTKCCQLGEDRRMACSEGYLSIVIHDTCRKQETTPINDNVSQCCSQLYANRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGA

SEQ ID NO: 19 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 20 - protein, 584 aa, artificial sequence
  N terminal is residues 1 to 572 of HSA. C terminal is residues 573 to 584 of Macaque albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKFVAASQAALA

SEQ ID NO: 21 - protein, 584 aa, Artificial Sequence
  N terminal is residues 1 to 572 from HSA. C terminal is residues 573 to 584 from mouse albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPNLVTRCKDALA

SEQ ID NO: 22 - protein, 584 aa, Artificial Sequence
  N terminal is residues 1 to 572 of HSA. C terminal is residues 573 to 584 of rabbit albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVESSKATLG

SEQ ID NO: 23 - protein, 584 aa, Artificial Sequence
  N-terminal is residues 1 to 572 of HSA. C-terminal is residues 573 to 583 of sheep albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVASTQAALA

SEQ ID NO: 24 - protein, 399 aa, Artificial sequence
  Artificial albumin variant human serum albumin domain 1 and human serum albumin domain 3
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 25 - protein, 403 aa, Artificial sequence
  Artificial albumin variant human serum albumin domain 2 and human serum albumin domain 3
DELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 26 - protein, 410 aa, Artificial sequence
  Artificial albumin variant two consecutive copies of human serum albumin domain 3
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 27 - protein, 205 aa, Artificial sequence [sequence omitted]

SEQ ID NO: 28 - protein, 615 aa, Artificial Sequence
  HSA Domain III + HSA Domain III + HSA Domain III
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 29 - protein, 604 aa, Artificial Sequence
  HSA Domain I + HSA Domain III + HSA Domain III
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 290 aa, Homo sapiens
  misc_feature (1)..(290): Truncated heavy chain of the major histocompatibility complex class I-like Fc receptor (FCGRT) (together, SEQ ID No. 30 and SEQ ID No. 31 form FcRN)
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVEL

SEQ ID NO: 31 - protein, 119 aa, Homo sapiens
  misc_feature (1)..(119): Beta-2-microglobulin (together, SEQ ID No. 30 and SEQ ID No. 31 form FcRN)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 32 - protein, 585 aa, Artificial Sequence
  N-terminus of Macaque albumin, C-terminus of HSA (Mac-HSAC)
DTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGKKLVAASQAALGL

SEQ ID NO: 33 - protein, 584 aa, Artificial Sequence
  N-terminus of Macaque albumin, C-terminus of Mouse albumin (Mac-MouseC)
DTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPNLVTRCKDALA

SEQ ID NO: 34 - protein, 584 aa, Artificial Sequence
  N-terminus of Macaque albumin, C-terminus of Rabbit albumin (Mac-RabC)
DTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKLVESSKATLG

SEQ ID NO: 35 - protein, 584 aa, Artificial Sequence
  N-terminus of Macaque albumin, C-terminus of Sheep albumin (Mac-SheepC)
DTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKLVASTQAALA

SEQ ID NO: 36 - DNA, 1780 bp, Artificial Sequence
  Synthetic sequence encoding macaque albumin and the 5′ region of the mADHt
agatctttggataagagagatacgcacaaaagcgaagttgcacacagattcaaagacttgggagaggaacatttcaagggtctagtactggttgcattttcccaatacttgcaacagtgtccatttgaagagcatgtaaaactggtgaacgaagttactgagtttgctaagacatgtgttgcagatgagtcagcagaaaactgtgataagtctttacacaccttgttcggggataaactatgcactgttgctactttacgtgaaacatatggcgaaatggcggattgctgtgccaagcaagagccagaaaggaacgagtgtttccttcaacataaagacgataatccaaacctccctcctctggtcagaccagaggttgatgtcatgtgtacagcttttcatgataacgaagcaactttcctcaaaaagtatttgtatgaggtagctagacgtcacccatacttctacgctccagagttactctttttcgcggctagatacaaggccgcgttcgctgaatgctgtcaggcagctgacaaggctgcatgcttattgcctaagttagatgagctaagagatgaaggtaaagcatctagtgctaagcaaagattgaaatgcgcatctcttcaaaagttcggtgatagagctttcaaagcctgggcagtggctcgattatcacaaaagtttcctaaggcagaattcgccgaagtcagcaagttagtcactgacctaacaaaggttcatacagagtgttgtcatggtgatctattggaatgtgcggacgatagagccgacttagccaaatacatgtgtgaaaatcaagattcaatctcttctaagttgaaggaatgttgtgataagcctctattggagaaatcacattgtttggccgaagttgaaaatgatgagatgccagctgatctaccatctctggcagcagattacgttgaatccaaagacgtctgcaaaaactacgctgaggctaaagacgttttccttggaatgtttttgtacgaatacgctagacgacatccagattactccgtcatgttgctgttaagattagctaaggcttatgaggcgacattggagaagtgttgtgctgccgctgatcctcatgaatgctatgccaaggtttttgatgaattccaacctcttgttgaagagccacagaatcttgtaaaacagaactgcgaactgtttgaacaactaggtgaatacaaattccaaaatgccttgttagtgaggtataccaaaaaggttcctcaagtgagtactccaacattagttgaagtctctagaaatctcggcaaagtgggagctaagtgctgtaagttgcctgaggcaaaacgtatgccttgtgcagaagattacttgtccgtggttctaaacagactttgtgttttgcatgaaaagactccagtatcagaaaaagtaacaaagtgctgcaccgagagtttagttaacagaaggccatgtttctctgccctagaattggatgaggcctatgtcccaaaggctttcaatgcagaaactttcacgtttcacgccgatatgtgtacactctctgaaaaggaaaaacaagtcaaaaagcaaactgctttagtagaactagtaaaacacaagccaaaagcgaccaaggaacagcttaagggtgtcatggataacttcgcagccttcgtggaaaagtgttgcaaggctgatgacaaggaagcatgttttgcagaggaagggccaaagtttgttgcagcttcacaggccgcattagcctaataagctt

SEQ ID NO: 37 - DNA, 2059 bp, Artificial Sequence
  3′ region of PRB1 promoter, modified fusion leader sequence, nucleotide sequence encoding HSA and 5′ region of the modified ADH1 terminator
ggtctcaagctaacctaattctaacaagcaaagatgaagtgggttttcatcgtctccattttgttcttgttctcctctgcttactctagatctttggataagagagacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatccgcggaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccctcgagaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgccttaggtttgtaataagcttaattaatatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgc

SEQ ID NO: 38 - DNA, 92 bp, Artificial Sequence
  Oligonucleotide xAP317
attaattaagcttattataagcctaaggcagcttgacttgcagcaacaagttttttcccttcctctgcaaaacatgcttccttgtcatcagc

SEQ ID NO: 39 - DNA, 91 bp, Artificial sequence
  Oligonucleotide xAP318
atattaattaagcttattaagccaaagcatctttacatctagtaaccaaatttggcccttcctctgcaaaacatgcttccttgtcatcagc

SEQ ID NO: 40 - DNA, 91 bp, Artificial sequence
  Oligonucleotide xAP319
atattaattaagcttattaacccaaagtagctttagaagattcaaccaatttaggcccttcctctgcaaaacatgcttccttgtcatcagc

SEQ ID NO: 41 - DNA, 91 bp, Artificial sequence
  Oligonucleotide xAP320
atattaattaagcttattaagccaaagcagcttgagtagaagcaaccaatttaggcccttcctctgcaaaacatgcttccttgtcatcagc

SEQ ID NO: 42 - DNA, 27 bp, Artificial sequence
  Oligonucleotide xAP321
ctttagtagaactagtaaaacacaagc

SEQ ID NO: 43 - DNA, 17 bp, Artificial sequence
  Oligonucleotide xAP265
gctcgcctgagccagag

SEQ ID NO: 44 - DNA, 30 bp, Artificial sequence
  Oligonucleotide xAP294
gaattaagcttattattagcctaaggcagc

SEQ ID NO: 45 - DNA, 30 bp, Artificial sequence
  Oligonucleotide xAP295
gaattaagcttattataattataaggcagc

SEQ ID NO: 46 - DNA, 33 bp, Artificial sequence
  Oligonucleotide xAP296
gaattaagcttattataagccttaggcagcttg

SEQ ID NO: 47 - DNA, 39 bp, Artificial sequence
  Oligonucleotide xAP297
gaattaagcttattataagcctaattaggcttgacttgc

SEQ ID NO: 48 - DNA, 48 bp, Artificial sequence
  Oligonucleotide xAP298
gaattaagcttattataagcctaaggcttattgacttgcagcaacaag

SEQ ID NO: 49 - DNA, 48 bp, Artificial sequence
  Oligonucleotide xAP299
gaattaagcttattataagcctaaggcagcttaacttgcagcaacaag

SEQ ID NO: 50 - DNA, 48 bp, Artificial sequence
  Oligonucleotide xAP300
gaattaagcttattataagcctaaggcagcttgttatgcagcaacaag

SEQ ID NO: 51 - DNA, 48 bp, Artificial sequence
  Oligonucleotide xAP301
gaattaagcttattataagcctaaggcagcttgactttaagcaacaag

SEQ ID NO: 52 - DNA, 56 bp, Artificial sequence
  Oligonucleotide xAP302
gaattaagcttattataagcctaaggcagcttgacttgcttaaacaagttttttac

SEQ ID NO: 53 - DNA, 56 bp, Artificial sequence
  Oligonucleotide xAP303
gaattaagcttattataagcctaaggcagcttgacttgcagcttaaagttttttac

SEQ ID NO: 54 - DNA, 60 bp, Artificial sequence
  Oligonucleotide xAP304
gaattaagcttattataagcctaaggcagcttgacttgcagcaacttattttttaccctc

SEQ ID NO: 55 - DNA, 60 bp, Artificial sequence
  Oligonucleotide xAP305
gaattaagcttattataagcctaaggcagcttgacttgcagcaacaagttatttaccctc

SEQ ID NO: 56 - DNA, 63 bp, Artificial sequence
  Oligonucleotide xAP306
gaattaagcttattataagcctaaggcagcttgacttgcagcaacaagttttttttactcctc

SEQ ID NO: 57 - DNA, 74 bp, Artificial sequence
  Oligonucleotide xAP307
gaattaagcttattataagcctaaggcagcttgacttgcagcaacaagttttttaccctcctcggcttagcagg

SEQ ID NO: 58 - DNA, 28 bp, Artificial sequence
  Oligonucleotide xAP314
ctcaagaaacctaggaaaagtgggcagc